SEQUENCE LISTING (34 sequences)

SEQ ID NO: 1 - DNA, 942 bp, Arabidopsis thaliana
atgtcatcgtcaaattggatcgacgacgcttttacagaggaagagcttctcgctatcgacgccatcgaagcttcctacaatttctcccgttcttcttcttcttcttcctctgctgctccgaccgtacaagctacaacctccgtccatggccacgaggaggatccaaatcaaatccccaataatatccgtcgccaattgcctcgttccatcacttcttctacatcttataaacgatttcctctctcccgttgccgagctaggaattttccagcaatgaggtttggtggtaggattttgtatagcaagactgctactgaggttgataagcgagcaatgcagcttattaaagttcttgataccaagagagatgaatctggaatagcttttgttggcttggatattgagtggagaccaagttttagaaaaggtgttctcccggggaaggttgcgactgtccagatatgtgtagatagtaattattgtgatgttatgcatatttttcattctggtatccctcaaagtctccaacatcttattgaagattcaacacttgtaaaggtaggtattggaattgatggtgactctgtgaagcttttccatgactatggagttagtatcaaagatgttgaggatctttcagatttagccaaccaaaaaattggtggagataaaaaatggggccttgcctcactaactgagacacttgtttgcaaagagctcctgaagccaaacagaatcaggcttgggaactgggagttttatcctctgtcaaagcagcagttacaatacgcagcaacggatgcttatgcttcatggcatctttacaaggtaacaacaacgaaaaaccatcttctcacactcaacgaccttgaagcaaaaatctcacatcgttctaattataatactgttacttgtcgaaaacctggaggttatcttcggtga

SEQ ID NO: 2 - protein, 313 aa, Arabidopsis thaliana
MSSSNWIDDAFTEEELLAIDAIEASYNFSRSSSSSSSAAPTVQATTSVHGHEEDPNQIPNNIRRQLPRSITSSTSYKRFPLSRCRARNFPAMRFGGRILYSKTATEVDKRAMQLIKVLDTKRDESGIAFVGLDIEWRPSFRKGVLPGKVATVQICVDSNYCDVMHIFHSGIPQSLQHLIEDSTLVKVGIGIDGDSVKLFHDYGVSIKDVEDLSDLANQKIGGDKKWGLASLTETLVCKELLKPNRIRLGNWEFYPLSKQQLQYAATDAYASWHLYKVTTTKNHLLTLNDLEAKISHRSNYNTVTCRKPGGYLR

SEQ ID NO: 3 - DNA, 1929 bp, Arabidopsis thaliana
atgagatttgatgatcccatggatgagttcaagaggaatcgaaagatggaggaagattcgaagaaggtaatcgatgtgaaagtggctgagagtgataagggattcgcgaaatttggcaaggcagaggttccgtttcatataccgacgttaacgaagcctcaagaggagtataagattttggtagacaatgctaataatccttttgagcatgttttgttggagaagagtgaagacggtcttcggttcattcatccactggaggaactatctgtgatggactttgttgatagaaatctaagtgagatgagacctgttaagcctctcccattggaagagactccattcaagctagttgaagaagtcaaagatcttgaggacttagctgctgcattgcaaagtgttgaagagtttgctgtcgatctggagcataatcagtatagaacttttcaaggattaacatgcttgatgcaaatctctactagaaccgaggattatattgttgatatattcaagctttgggatcacattggtccttatctaagggaactcttcaaagaccctaaaaagaaaaaggtaatccatggagcagatcgagatattatttggcttcaacgggactttggcatttatgtctgcaatctttttgacacaggacaggcttcaagggtgctaaagctggagagaaatagtctggaatttcttctgaagcattattgtggagttgctgcaaacaaagaataccaaaaagcagactggagaataagaccccttccagatgtaatgaaaagatatgctagagaagatacacattatcttttgtacatttatgatgtaatgcgaatggagttgcacacaatggcaaaggaagatgagcaatctgactctcctctggtagaggtgtacaagcgcagttatgacgtgtgcatgcaactatatgaaaaagagctttggactagggattcatatcttcacgtttatggggttcagacaggtaatctcaatgcggttcaactttccattgttgcgctgcaggggctttgtgaatggcgggatcggattgcacgcgcagatgatgagagcaccggttatgtattgccaaataaaactctttttgacatagccaaggagatgccaattgttgttgcccagttgcgccgtttgttgaagtcaaagcttccttacctcgagcgtaattttgacgcagtgatcagtgtcatcagacgatcaatgcaaaatgcagcggcattcgagccagttgttcaatctttgaaagataggcgtcctgaaacagtggttgaaatgaatatagaacctaagattgagaaaacagacacaggagcttcagcgtcttctctgagtctggagaaggtttgtgtggatgattcaaagaaacaaagcagtggttttggagttttgccgttaaagaggaagttggaaagtgacaaaacggtggttgaaaagaatatcgaacctaagattgagaaaacaggcacagaagcttcagcttcttctctgagttcgaagaaggtttgtgtggatgattcaaagaaacaaagcagtggttttggagttttgctgtcaaagaggaagtttgaaagtgataacaagaagttgcaggtaaaagaagaggtcaaagtgtccaagtccaagccagataaggtaatcatagtggtggatgatgatgatgatgatgatgatgatgagtcttatgaacagagcacgaaagccgctgatgctttggacagagtttcggaaacgccttcgaagggatcaccatcgttgactcaaaagccgaagacatgtaatacagaggttattgtgttagacgatgatgatgactcggaaagcagagaagatgaagacatgcgtaggagaagtgagaaacataggagattcatgaatatgaaacgtggctttcttaacatttag

SEQ ID NO: 4 - protein, 642 aa, Arabidopsis thaliana
MRFDDPMDEFKRNRKMEEDSKKVIDVKVAESDKGFAKFGKAEVPFHIPTLTKPQEEYKILVDNANNPFEHVLLEKSEDGLRFIHPLEELSVMDFVDRNLSEMRPVKPLPLEETPFKLVEEVKDLEDLAAALQSVEEFAVDLEHNQYRTFQGLTCLMQISTRTEDYIVDIFKLWDHIGPYLRELFKDPKKKKVIHGADRDIIWLQRDFGIYVCNLFDTGQASRVLKLERNSLEFLLKHYCGVAANKEYQKADWRIRPLPDVMKRYAREDTHYLLYIYDVMRMELHTMAKEDEQSDSPLVEVYKRSYDVCMQLYEKELWTRDSYLHVYGVQTGNLNAVQLSIVALQGLCEWRDRIARADDESTGYVLPNKTLFDIAKEMPIVVAQLRRLLKSKLPYLERNFDAVISVIRRSMQNAAAFEPVVQSLKDRRPETVVEMNIEPKIEKTDTGASASSLSLEKVCVDDSKKQSSGFGVLPLKRKLESDKTVVEKNIEPKIEKTGTEASASSLSSKKVCVDDSKKQSSGFGVLLSKRKFESDNKKLQVKEEVKVSKSKPDKVIIVVDDDDDDDDDESYEQSTKAADALDRVSETPSKGSPSLTQKPKTCNTEVIVLDDDDDSESREDEDMRRRSEKHRRFMNMKRGFLNI

SEQ ID NO: 5 - DNA, 714 bp, Arabidopsis thaliana
atgaatttgcattttgatttttggtgttttatatttgaaactaatgcagagaaaccttcgaatggtcatccatatgaaactgagatcactgttttgttagagaatcctcagattgagtttggatttttgagaggagagtgttcattggaaatgagtgattcatatgtgtgggttgagacagagtcgcagttaaaggaacttgcagaaatattagcaaaagaacaagtttttgcggttgacactgagcagcatagtttgcggtcgtttcttggtttcactgctctaattcagatttctacacatgaggaagactttttggtggacacaattgcgttacatgatgtaatgagtattcttcgtcctgttttctctgatcctaatatttgtaaggtgtttcacggggctgacaacgatgttatctggcttcaaagagacttccatatatatgttgttaatatgtttgatactgccaaggcatgtgaagtgttgtcaaagcctcaacgatcactggcatacttacttgagacagtatgtggagtggctactaacaaattgctgcagcgtgaagattggagacagcgtcctctgtccgaagagatggtgcgatatgctagaacagatgcacactatctgctttatattgcagatagtttgacaactgaactcaaacaattagccactggtaggcatctttgctatggagaaacattttag

SEQ ID NO: 6 - protein, 237 aa, Arabidopsis thaliana
MNLHFDFWCFIFETNAEKPSNGHPYETEITVLLENPQIEFGFLRGECSLEMSDSYVWVETESQLKELAEILAKEQVFAVDTEQHSLRSFLGFTALIQISTHEEDFLVDTIALHDVMSILRPVFSDPNICKVFHGADNDVIWLQRDFHIYVVNMFDTAKACEVLSKPQRSLAYLLETVCGVATNKLLQREDWRQRPLSEEMVRYARTDAHYLLYIADSLTTELKQLATGRHLCYGETF

SEQ ID NO: 7 - DNA, 849 bp, Arabidopsis thaliana
atgcagattgcattctctaatgcaatatacttggttgatgtcatcgaaggtggagaggtgattatgaaagcgtgtaagcctgcactcgagtctaattacatcacgaaagttattcacgattgcaagcgtgacagtgaggctctatacttccagtttgggataagattgcacaatgttgtggacactcagattgcttattctctgattgaagaacaagaagggcggaggagacctctagatgattacatatcgtttgtttcactccttgctgatccacgttactgcggtatatcctatgaagagaaagaagaagttcgagttctcatgcgccaggacccaaagttttggacatacaggcctatgactgagctcatgatccgcgcagctgctgatgatgtccgcttccttctgtatctctatcacaaaatgatgggaaagctaaatcagcggtcactatggcatcttgcagttcgtggtgctttgtactgtcggtgtctctgctgcatgaatgatgctgattttgctgattggccaaccgttcctccaattccagttttcctcgttaaggtcgtatatgctgtagagacaaagaaaaaaagacgggtgacattagcttcgattgggttactgattgtagttggacttttaaatgtggcagataacctgaagtcagaagatcaatgtcttgaagaagagatcctgtcagtgcttgatgttccaccaggaaagatgggacgtgtgattggaaggaaaggagcatcgatcctcgccattaaggaagcttgcaacgcggaaattctaattggaggggcaaagggtccacctgataaggttagtcttattccatag

SEQ ID NO: 8 - protein, 282 aa, Arabidopsis thaliana
MQIAFSNAIYLVDVIEGGEVIMKACKPALESNYITKVIHDCKRDSEALYFQFGIRLHNVVDTQIAYSLIEEQEGRRRPLDDYISFVSLLADPRYCGISYEEKEEVRVLMRQDPKFWTYRPMTELMIRAAADDVRFLLYLYHKMMGKLNQRSLWHLAVRGALYCRCLCCMNDADFADWPTVPPIPVFLVKVVYAVETKKKRRVTLASIGLLIVVGLLNVADNLKSEDQCLEEEILSVLDVPPGKMGRVIGRKGASILAIKEACNAEILIGGAKGPPDKVSLIP

SEQ ID NO: 9 - DNA, 720 bp, Arabidopsis thaliana
atggctaggatcagaagaagaatccaaaagcgccatatccacgaaaaccgctacatcgatttctttggagaacgtttgatcgtcacggtcactcatactacctcaaccatccgccgttggattcatagcatccgtttcttcagccgtcttcgctcctcacaccctctcgttgttggactcgacgtccaatggacacccggtggttccgatcctccaccggatattctccaactatgtgttggtaaccgctgtctcatcatccagttgtctcactgtaaacgcattcctgaggtccttcgaagtttcttggaagatgagacaatcacttttgtcggcgtctggaacagccaagaccagggcaagctcgaaagattccgccatcagttggagatatggagacttctagacataaggcactatctgcctacgaggctcctcaatagttcgtttgagaagattgtagaggagtgtttggggtacaagggagtgaggaaagataaggagatatgtatgagtaattggggtgctcgtagcctttcccatgatcagattgttcaggcgtcagatgatgtctatgtttgctgcaagctcggtgttaaggaatgtatctggaaagagcgctcgaatgttaaagaacgtatctggaaagagagctcgaatgttaaggaacatatctggaaagagagctcgaaactttattttgttggggtatgtttctga

SEQ ID NO: 10 - protein, 239 aa, Arabidopsis thaliana
MARIRRRIQKRHIHENRYIDFFGERLIVTVTHTTSTIRRWIHSIRFFSRLRSSHPLVVGLDVQWTPGGSDPPPDILQLCVGNRCLIIQLSHCKRIPEVLRSFLEDETITFVGVWNSQDQGKLERFRHQLEIWRLLDIRHYLPTRLLNSSFEKIVEECLGYKGVRKDKEICMSNWGARSLSHDQIVQASDDVYVCCKLGVKECIWKERSNVKERIWKESSNVKEHIWKESSKLYFVGVCF

SEQ ID NO: 11 - DNA, 654 bp, Arabidopsis thaliana
atgaagagaggtatcaaacatctatgtttcaatggcttcacgggctactcatcacttcatcatcattatcatgaacaccacgtcgacttctttggagaacgtttgatcgtcacagtcactcatactccctcagtgatacgtcgatggatccacagtatccgcttcgtcagccgtcttcgcttatcacaccctctagttgtcggacttggcgttcaatggacaccccgtggttccgatcctccaccggatattctccaactatgtgttggtactcgctgtctcatcattcagttgtctcactgtaagtacgtccccgacgtccttagaagtttcttggaagatcagacaatcacttttgtcggcgtatggaacagccaagacaaggacaagctcgagagattccaccatcagttggatatctggagacttgtccacataaggcactatctccatccgttgctcttgagtagctcgtttgagacgattgtgaaggtgtatttggggcatgaaggagtgacgaaagataaggagttatgtatgagtaattggggtgctcgtagcctctctcatgatcagatagtacaagcgtcacatgatgtctatgtttgctgcaagctcggtgttaaggaacgtctctggaaaatgggagcttaa

SEQ ID NO: 12 - protein, 217 aa, Arabidopsis thaliana
MKRGIKHLCFNGFTGYSSLHHHYHEHHVDFFGERLIVTVTHTPSVIRRWIHSIRFVSRLRLSHPLVVGLGVQWTPRGSDPPPDILQLCVGTRCLIIQLSHCKYVPDVLRSFLEDQTITFVGVWNSQDKDKLERFHHQLDIWRLVHIRHYLHPLLLSSSFETIVKVYLGHEGVTKDKELCMSNWGARSLSHDQIVQASHDVYVCCKLGVKERLWKMGA

SEQ ID NO: 13 - DNA, 261 bp, Arabidopsis thaliana
atgatcaagtcgatcgagagctttattgctcgttatgttttccaagctacattatacacaatctggtgcgaacgaaatggcagaaggcatggagagagagaggccgaaccagccgctcttctaataaaattgattgataagcaagccatctttcctcaatcagagcaactgatgatctacgctatgacaagagcctccaaatctggtttgcatcgagtggttgaaatcaaaccatctatagttcatgtgtttaacctttaa

SEQ ID NO: 14 - protein, 86 aa, Arabidopsis thaliana
MIKSIESFIARYVFQATLYTIWCERNGRRHGEREAEPAALLIKLIDKQAIFPQSEQLMIYAMTRASKSGLHRVVEIKPSIVHVFNL

SEQ ID NO: 15 - DNA, 2733 bp, C. elegans
atggaagaagaaccgtacaaaagaaagctaacgaaagccgagaaaaaggcaaaatatcgaacagactacgccgaaccgttgaagtcacgccgggaagttctgaaagcaattatgaatggacccgagtctgagcgggagagaaaagttcgagccaaaaatcgagaatttttcaacgaggactatagatcaggagtcaacatctacggaatggctgtggatatgatgaaagcgatgccggatagaggaaaaacatcgggacaaagtttggcggtttggtatctggaggattttggagtttggttaaaagagtcgggacaggagacggagctcagacagaaatatctgactggaactattcaaataaacgccttagatgtgtgcacaattggacaaaagcagcttctcagtgaaatcttcgatatcaccaaagagaaattcactgaggacattacacagttgctagatgctgccatcaagaaacaagacttctccgttgctgcagatatggctattcagtacaatcttctacgggatcatcatttcgaacatcttgttcttccattaatgctatctggcaaagatcaaacggcttataaattgataagtaacaatgagaggatgcaacagcagcttgtagagttttttgatcgaatggttggaatctcagtggttgccgttgaagagatgctgaaaccctacaaggaaaccaaaattatgacgattcctatggagaaattgacgggaaaaacgttggacaaactcatttcgacgattatcaacaaaaatactcacgaatacaacttctccagggaattgtcgaagttcgccaaaaaccactcacagaatgggaatctgaaggcattgaagtttaatatcagtgaacgatacgagaagggaaaatccgatgacaactatttccagcatatggttgaaacttttaccaaagccgaagatgttcgtgaacctattttgttttacttgtggagctcaaatgacaccgagaaacaaatagatgccatctgctttgctatctacttaggaatcgctagttccagcagctatcaactgccgaatgttatgagggacttctttcgacaacctgattcgaagctcagagaagcaaaagaacttctagtgagaagaaaaacactgcaagttcctctaaatggcgaacaattattcgtatttgagaatgagcgaagaactcaaatccacatggtgaaaactgaatctgagatgaattacttatgttccgagatcaaatcactaagcgacgagccagcacctgtttacgttggattcgattctgagtggaaaccgtcgaatcttacagctgttcatgactcgaaaattgctatcatacaattgttcttcaaaaattgtgtatggcttgtggattgcgtagaattagaaaaggcaaatatggcagatgactggtggcaaaagttcgcatctcgattgttcggagattctcctgtaaaagtcgtaggatttgatatgaggaacgatctggatgcaatggctacaatcccagcactgaagtcatccatgaagatagaagataccaaaaatgcattcgatctgaagcgattagcagagaatgtttgcgatatcgacatggaaattttagagctgccaaagaagactttcaaattggcagatttgacacattatctactgggattggagctcgacaaaactgaacaatgcagtaactggcaatgtcgtcctctgcgaaaaaaacaaattgtgtacgcagcattggacgcagttgtcgtggtggaaacattcaagaaaatcttgtcgattgtagaggagaaaaacaaggacgcagatatcgagaagattgtcagagaatcaaatgtaatggctccgaaaaaagacaaaggacacaaatcgtaccgcaagctgaaaactattccatggcttgagctctacgatatcttgcgaagccatcgtaatcctacgagatcaccacagcgaccacacgacattaaagttattgttgacacaatgctgattggatttggaaagaatctgaggagagttggaattgatgttattcttccaaaagacgtgagcgacttccgaaagtacctgaaggaaattgaacgagttggcggcgagcatctacgtcatataatcacagtgccatcgaaaagttacgaagccttgaaaatggattatgataattatacaattgcaattccggaactcaataacatgtctcccgtagatcagcttattgagtttttcgacctgttcaacgtagatattcgtccggaagacgtatatcctcgatgcactgaatgcaattctcggcttcaaattaaattccccggaccagttttacatttcttgcaccaatactgtgtcatccatgtgcaaaatgtttatcgtgcggatatgagcgaatttccactggaagaatggtggaatcgtatgcttcatatcaatccagacgactacgacggagtaaaagtggaaatgtcgcgaccatctccaacaagcaagtggatcgtggcaactgttcccacaggatgcctacatattacgcgacaaactgcacttcacaccaatctgccagatggaattgaagttcgaatccacaaagtgcctgatgacgagttcaagcgtcgaaatctcagcttctatgtgtgtggagaatgcggtacggtggcttgtgatggtcgtggcaatcaagcgtcggagagcaccagccaggaatgttga

SEQ ID NO: 16 - protein, 910 aa, C. elegans
MEEEPYKRKLTKAEKKAKYRTDYAEPLKSRREVLKAIMNGPESERERKVRAKNREFFNEDYRSGVNIYGMAVDMMKAMPDRGKTSGQSLAVWYLEDFGVWLKESGQETELRQKYLTGTIQINALDVCTIGQKQLLSEIFDITKEKFTEDITQLLDAAIKKQDFSVAADMAIQYNLLRDHHFEHLVLPLMLSGKDQTAYKLISNNERMQQQLVEFFDRMVGISVVAVEEMLKPYKETKIMTIPMEKLTGKTLDKLISTIINKNTHEYNFSRELSKFAKNHSQNGNLKALKFNISERYEKGKSDDNYFQHMVETFTKAEDVREPILFYLWSSNDTEKQIDAICFAIYLGIASSSSYQLPNVMRDFFRQPDSKLREAKELLVRRKTLQVPLNGEQLFVFENERRTQIHMVKTESEMNYLCSEIKSLSDEPAPVYVGFDSEWKPSNLTAVHDSKIAIIQLFFKNCVWLVDCVELEKANMADDWWQKFASRLFGDSPVKVVGFDMRNDLDAMATIPALKSSMKIEDTKNAFDLKRLAENVCDIDMEILELPKKTFKLADLTHYLLGLELDKTEQCSNWQCRPLRKKQIVYAALDAVVVVETFKKILSIVEEKNKDADIEKIVRESNVMAPKKDKGHKSYRKLKTIPWLELYDILRSHRNPTRSPQRPHDIKVIVDTMLIGFGKNLRRVGIDVILPKDVSDFRKYLKEIERVGGEHLRHIITVPSKSYEALKMDYDNYTIAIPELNNMSPVDQLIEFFDLFNVDIRPEDVYPRCTECNSRLQIKFPGPVLHFLHQYCVIHVQNVYRADMSEFPLEEWWNRMLHINPDDYDGVKVEMSRPSPTSKWIVATVPTGCLHITRQTALHTNLPDGIEVRIHKVPDDEFKRRNLSFYVCGECGTVACDGRGNQASESTSQEC

SEQ ID NO: 17 - DNA, 4299 bp, Homo sapiens
atgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgtacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaacttaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagtttcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagttaa

SEQ ID NO: 18 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKFLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 19 - DNA, 30 bp, Artificial Sequence
  1...30
cgacatgatctgatacatcgttatgccatt

SEQ ID NO: 20 - DNA, 29 bp, Artificial Sequence
  1...29
cattttataataacgctgcggacatctac

SEQ ID NO: 21 - DNA, 1041 bp, Arabidopsis thaliana
atgtttgagtttttcgcttcaggaggaaggtcgccgacacaagaagctaatgagccaccagttccgatttacattgtgacggatccgtttcaacttcctgctgatttcctaaacccttctcctgaaaagaaattggttatcggttttgactgtgagggtgttgacctctgccgacatgggaaactttgtatcatgcagattgcattctctaatgcaatatacttggttgatgtcatcgaaggtggagaggtgattatgaaagcgtgtaagcctgcactcgagtctaattacatcacgaaagttattcacgattgcaagcgtgacagtgaggctctatacttccagtttgggataagattgcacaatgttgtggacactcagattgcttattctctgattgaagaacaagaagggcggaggagacctctagatgattacatatcgtttgtttcactccttgctgatccacgttactgcggtatatcctatgaagagaaagaagaagttcgagttctcatgcgccaggacccaaagttttggacatacaggcctatgactgagctcatgatccgcgcagctgctgatgatgtccgcttccttctgtatctctatcacaaaatgatgggaaagctaaatcagcggtcactatggcatcttgcagttcgtggtgctttgtactgtcggtgtctctgctgcatgaatgatgctgattttgctgattggccaaccgttcctccaattccagttttcctcgttaaggtcgtatatgctgtagagacaaagaaaaaaagacgggtgacattagcttcgattgggttactgattgtagttggacttttaaatgtggcagataacctgaagtcagaagatcaatgtcttgaagaagagatcctgtcagtgcttgatgttccaccaggaaagatgggacgtgtgattggaaggaaaggagcatcgatcctcgccattaaggaagcttgcaacgcggaaattctaattggaggggcaaagggtccacctgataaggttagtcttattccatag

SEQ ID NO: 22 - protein, 346 aa, Arabidopsis thaliana
MFEFFASGGRSPTQEANEPPVPIYIVTDPFQLPADFLNPSPEKKLVIGFDCEGVDLCRHGKLCIMQIAFSNAIYLVDVIEGGEVIMKACKPALESNYITKVIHDCKRDSEALYFQFGIRLHNVVDTQIAYSLIEEQEGRRRPLDDYISFVSLLADPRYCGISYEEKEEVRVLMRQDPKFWTYRPMTELMIRAAADDVRFLLYLYHKMMGKLNQRSLWHLAVRGALYCRCLCCMNDADFADWPTVPPIPVFLVKVVYAVETKKKRRVTLASIGLLIVVGLLNVADNLKSEDQCLEEEILSVLDVPPGKMGRVIGRKGASILAIKEACNAEILIGGAKGPPDKVSLIP

SEQ ID NO: 23 - DNA, 1049 bp, Arabidopsis thaliana
accaaagcattaatttttatttttttgtttcagtaaaagaaatgtcatcgtcaaattggatcgacgacgcttttacagaggaagagcttctcgctatcgacgccatcgaagcttcctacaatttctcccgttcttcttcttcttcttcctctgctgctccgaccgtacaagctacaacctccgtccatggccacgaggaggatccaaatcaaatccccaataatatccgtcgccaattgcctcgttccatcacttcttctacatcttataaacgatttcctctctcccgttgccgagctaggaattttccagcaatgaggtttggtggtaggattttgtatagcaagactgctactgaggttgataagcgagcaatgcagcttattaaagttcttgataccaagagagatgaatctggaatagcttttgttggcttggatattgagtggagaccaagttttagaaaaggtgttctcccggggaaggttgcgactgtccagatatgtgtagatagtaattattgtgatgttatgcatatttttcattctggtatccctcaaagtctccaacatcttattgaagattcaacacttgtaaaggtaggtattggaattgatggtgactctgtgaagcttttccatgactatggagttagtatcaaagatgttgaggatctttcagatttagccaaccaaaaaattggtggagataaaaaatggggccttgcctcactaactgagacacttgtttgcaaagagctcctgaagccaaacagaatcaggcttgggaactgggagttttatcctctgtcaaagcagcagttacaatacgcagcaacggatgcttatgcttcatggcatctttacaaggttcttaaggaccttcctgatgctgtcagtggctcataacgtgaaggaggaagcttaaaggttagcctataaccccaagagttagcatcaaatgatatgatacacctaatctagtcaagtagatgcaattcttgtgaatattgtatctagttctggtccctttaaccgtccagaaactag

SEQ ID NO: 24 - protein, 288 aa, Arabidopsis thaliana
MSSSNWIDDAFTEEELLAIDAIEASYNFSRSSSSSSSAAPTVQATTSVHGHEEDPNQIPNNIRRQLPRSITSSTSYKRFPLSRCRARNFPAMRFGGRILYSKTATEVDKRAMQLIKVLDTKRDESGIAFVGLDIEWRPSFRKGVLPGKVATVQICVDSNYCDVMHIFHSGIPQSLQHLIEDSTLVKVGIGIDGDSVKLFHDYGVSIKDVEDLSDLANQKIGGDKKWGLASLTETLVCKELLKPNRIRLGNWEFYPLSKQQLQYAATDAYASWHLYKVLKDLPDAVSGS

SEQ ID NO: 25 - DNA, 22 bp, Artificial sequence
  misc_feature (1)..(22): Primer
ttcggaaccaccatcaaacagg

SEQ ID NO: 26 - DNA, 22 bp, Artificial Sequence
  Misc_feature (1)..(22): Primer
ttgctgcaactctctcagggcc

SEQ ID NO: 27 - DNA, 21 bp, Artificial Sequence
  Misc_feature (1)..(21): Primer
tcagctgttgcccgtctcact

SEQ ID NO: 28 - DNA, 16 bp, Artificial Sequence
  Misc_feature (1)..(16): n=a, c, g, or t
wgtgnagwancanaga

SEQ ID NO: 29 - DNA, 27 bp, Artificial Sequence
  Misc_feature (1)..(27): Primer
gctccgcccacataattcaaacaacac

SEQ ID NO: 30 - DNA, 22 bp, Artificial Sequence
  Misc_feature (1)..(22): Primer
ttcgaaaacattacctccgatc

SEQ ID NO: 31 - DNA, 25 bp, Artificial Sequence
  Misc_feature (1)..(25): Primer
ggcttttgcatttggtatctactag

SEQ ID NO: 32 - DNA, 25 bp, Artificial Sequence
  Misc_feature (1)..(25): Primer
atgtcatcgtcaaattggatcgacg

SEQ ID NO: 33 - DNA, 27 bp, Artificial Sequence
  Misc_feature (1)..(27): Primer
cgcttatcaacctcagtagcagtcttg

SEQ ID NO: 34 - DNA, 24 bp, Artificial Sequence
  Misc_feature (1)..(24): Primer
ttatgagccactgacagcatcagg

(19) United States
(12) Patent Application Publication
(10) Pub. No.: US 2006/0090218 A1
(43) Pub. Date: Apr. 27, 2006

(54) Method of controlling gene expression and gene silencing

(75) Inventors: Joshua Z. Levin, Silver Spring, MD (US); Kenneth L. Phillips, Durham, NC (US); Gregory Budziszewski, Durham, NC (US); Frederick Meins JR., Riehen (CH); Evgueni A. Glazov, Basel (CH)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 11/301,924

(22) Filed: Dec. 13, 2005

Related U.S. Application Data
(62) Division of application No. 09/896,186, filed on Jun. 29, 2001.
(60) Provisional application No. 60/222,202, filed on Aug. 1, 2000.

(51) Int. Cl. A01H 1/00; C12N 15/82

(52) U.S. Cl. 800/278; 435/468

(57) ABSTRACT

The present invention relates to methods to regulate gene expression in plants. In particular, manipulation of the expression in a plant cell of a nucleotide sequence encoding a polypeptide comprising a 3′-5′ exonuclease domain is disclosed. More stable and predictable expression is thus obtained. The present invention also relates to method of increasing or decreasing post-transcriptional silencing. The invention further relates to novel nucleic acid molecules comprising nucleotide sequences encoding polypeptides comprising a 3′-5′ exonuclease domain.

This case is a divisional application of U.S. patent application Ser. No. 09/896,186 and which claims benefit of U.S. Provisional Patent Application No. 60/222,202 filed Aug. 1, 2000, which are hereby incorporated by reference in their entireties.

### FIELD OF THE INVENTION

The present invention relates to the field of molecular biology, in particular to the regulation of gene expression in plants and to gene silencing. The present invention also relates to a novel isolated nucleic acid molecules comprising nucleotide sequences encoding novel polypeptides comprising a 3′-5′ exonuclease domain.

### BACKGROUND OF THE INVENTION

Developments in the techniques of molecular biology and transformation have allowed the production of transgenic plants with various desirable traits. However, in some transgenic lines, the loss of expression of previously active genes has been observed in response to developmental, environmental or unknown signals. This phenomenon is commonly referred to as gene silencing. It occurs at a frequency higher than that of mutations, yet is markedly stable during somatic transmission. Chromosomal position or structure of the affected loci are factors determining the frequency and strength of gene silencing and inactivation seems to preferentially affect genes present in multiple copies and is thought to be a consequence of sequence redundancy. While post-transcriptional silencing seems to mainly involve the formation of aberrant RNA molecules and is occasionally, but not necessarily, accompanied by DNA methylation, silencing that interferes with transcription initiation is more strictly correlated with hypermethylation of the DNA and possibly with alteration of chromatin structure at the silent loci. Currently, post-transcriptional gene silencing (PTGS) generally refers to the epigenetic inactivation of gene expression resulting from the specific degradation of mRNAs derived from genes with transcribed regions similar in sequence (Meins (2000) Plant Mol. Biol. 43: 261-273).

There have been attempts to understand the mechanism of gene silencing in plants. For example, in Arabidopsis two lines with independent mutant loci egs1 and egs2 were isolated from a M2 population by direct screening for silencing of an Agrobacterium rhizogenes rolB transgene (Dehio and Schell (1994) PNAS 91:5538-42). The egs1 mutation appears to lead to the inactivation of this rolB transgene, and consequently, the wild type egs1 allele may actively prevent silencing. Other mutants affected in post-transcriptional gene silencing (sgs1 and sgs2, for suppressor of gene silencing) have been described in Elmayan et al. (1998) Plant Cell 10:1747-58. In this case, mutant plants carried a recessive monogenic mutation that appears to be involved in the release of silencing. In yet another report, disruption of a gene called MOM released transcriptional silencing of methylated genes (Amedeo et al. (2000) Nature 405:203-206). Although promising, these results are still preliminary.

Recently, five RecQ-like proteins have been isolated and characterized from Arabidopsis thaliana (Hartung et al. (2000) Nucleic Acids Research 28:4275-4282). These proteins are proposed to be involved in processes linked to DNA replication, DNA recombination and gene silencing.

The cellular functions involved in the switch from active to inactive genes are still not known, and tools allowing one skilled in the art to manipulate this phenomenon are lacking. One such enzyme that is proposed to be involved are exonucleases. A recent review of exoribonuclease superfamilies analyzed the structure and phylogenetic distribution of known exoribonucleases (Zuo et al. (2001) Nucleic Acid Res. 29:1017-1026). The authors grouped the exoribonucleases into six superfamilies and various subfamilies. The article furthered proposed common motifs to be used to characterize newly-discovered enzymes.

In the production of transgenic plants with improved characteristics large numbers of independent transgenic lines have to be tested through several generations to ensure that they are not affected by gene silencing. This is time-consuming and very expensive. There is therefore a long-felt but unfulfilled need for novel methods allowing one to effectively and predictably control gene silencing in plant cells in order to obtain plants with improved properties in a cost-effective manner.

There is also a need in the field of functional genomics to provide cells or plants having no or insignificant levels of gene silencing so that analysis of gene functions can be performed more efficiently. By inhibiting or removing expression of genes responsible for gene silencing, the expression of genes of interest in functional genomics may be analyzed without the interference of gene silencing.

There is further a need in the field for increased gene silencing in cells or plants for more stringent control of gene expression or resistance to pathogens, in particular, viral pathogens.

### SUMMARY OF THE INVENTION

The present invention addresses the need for methods to reproducibly and predictably manipulate gene expression in a plant cell. In particular, the present invention addresses the need for stable and predictable expression of a nucleotide sequence in a plant cell. According to the present invention, this is achieved by manipulating the expression in a plant cell of a nucleotide sequence encoding a polypeptide 3′-5′ exonuclease domain. The present invention therefore provides a clear advantage over the prior art by reducing the number of transgenic lines which have to be screened until a suitable line is selected, and by providing stable and better controlled expression of a nucleotide sequence in the plant cell.

In one aspect, the present invention encompasses novel methods for controlling gene silencing in a plant cell. The present invention encompasses the suppression of gene silencing or the increase in gene silencing in plants. In a preferred embodiment, this is achieved by altering the expression in the plant cell of a nucleotide sequence encoding a polypeptide comprising a 3′-5′ exonuclease domain. In another embodiment, the nucleotide molecule encodes a polypeptide comprising exonuclease activity, preferably having 3′-5′ RNA exonuclease activity. Preferably, the polypeptide comprises a 3′-5′ exonuclease domain. More preferably, the 3′-5′ exonuclease domain is an RNase D related domain. In another preferred embodiment, the polypeptide is identical or substantially similar to SEQ ID NO:2, SEQ ID NO:4, SEQ ID NO:6, SEQ ID NO:22, SEQ ID NO:10, SEQ ID NO:12, SEQ ID NO:14, SEQ ID NO:16 SEQ ID NO:18, SEQ ID NO:24, SEQ ID NO:36 or SEQ ID NO:38. Preferably, the nucleotide sequence is identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:15 SEQ ID NO:17, SEQ ID NO:23, SEQ ID NO:35 or SEQ ID NO:37. Most preferably, the nucleotide sequence is identical or substantially identical to SEQ ID NO:23.

In another embodiment, the invention provides novel isolated and substantially purified polypeptides comprising, consisting of or having an amino acid sequence identical or substantially similar to SEQ ID NO:24.

In a preferred embodiment, the expression of a nucleotide sequence encoding a polypeptide comprising a 3′-5′ exonuclease domain is altered by altering its transcription or translation. Reduced expression is for example obtained by expressing in the plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15 SEQ ID NO:17, SEQ ID NO:23, SEQ ID NO:35 or SEQ ID NO:37 in sense orientation, or a portion thereof; or expressing in the plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11 SEQ ID NO:13, SEQ ID NO:23, SEQ ID NO:35 or SEQ ID NO:37 in anti-sense orientation, or a portion thereof; or expressing in the plant cell a sense RNA of a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11 SEQ ID NO:13, SEQ ID NO:23, SEQ ID NO:35 or SEQ ID NO:37 or a portion thereof, and an anti-sense RNA of said nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11 SEQ ID NO:13, SEQ ID NO:23, SEQ ID NO:35 or SEQ ID NO:37 or a portion thereof, wherein said sense and said anti-sense RNAs are capable of forming a double-stranded RNA molecule; or expressing in said plant cell a ribozyme capable of specifically cleaving a messenger RNA transcript encoded by a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23; SEQ ID NO:35 or SEQ ID NO:37 or modifying by homologous recombination in said plant cell at least one chromosomal copy of a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or of a regulatory region thereof; or expressing in said plant cell a zinc finger protein that is capable of binding to a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or to a regulatory region thereof; or introducing into said plant cell a chimeric oligonucleotide that is capable of modifying at least one chromosomal copy of a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or a regulatory region thereof. Preferably, the expression of the sequence is altered by insertional mutagenesis, point mutation or deletion mutagenesis of the genomic copy of a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or a regulatory region thereof. Alternatively, the sequence has a mutation due to rearrangement. Increased expression of a polypeptide comprising a 3′-5′ exonuclease domain is also within the scope of the present invention and is, for example, obtained by over-expressing in the plant cell a nucleotide sequence of the present invention.

In a further aspect, the present invention encompasses methods to alter the expression of a nucleotide sequence of interest in a plant cell, and methods to stabilize the expression of a nucleotide sequence of interest in a plant cell. In a preferred embodiment, the nucleotide sequence of interest is a heterologous nucleotide sequence. In another preferred embodiment, the nucleotide sequence of interest is an endogenous nucleotide sequence of the plant cell. The expression of a nucleotide sequence of interest is preferably altered by altering the expression in the plant cell of a nucleotide sequence encoding a polypeptide comprising a 3′-5′ exonuclease domain as described above. The plant cell with altered expression of a nucleotide sequence encoding a polypeptide comprising a 3′-5′ exonuclease domain also comprises the nucleotide sequence of interest, or a portion thereof, or a reverse complement thereof. In a preferred embodiment, the nucleotide sequence of interest, or a portion thereof, or a reverse complement thereof is introduced into plant cell with altered expression of a nucleotide sequence encoding a polypeptide comprising a 3′-5′ exonuclease domain.

In a preferred embodiment, the nucleotide sequence of interest is derived from a pathogen of a plant, or is substantially similar thereto. A pathogen is, for example but not limited to, a viral, fungal or bacterial pathogen of a plant. Preferably the pathogen is a viral pathogen. Therefore, it is a further aspect of the present invention to provide for methods to control a pathogen comprising the steps of obtaining a plant cell with altered expression of a nucleotide sequence that encodes a polypeptide comprising a 3′-5′ exonuclease domain as described above and wherein the plant cell further comprises a nucleotide sequence identical or substantially similar to a nucleotide sequence derived from the pathogen.

The present invention also encompasses a recombinant nucleic acid molecule comprising a nucleotide sequence that encodes a polypeptide comprising a 3′-5′ exonuclease domain as described above, or a reverse complement thereof, or complement thereof.

The present invention also encompasses an expression cassette comprising a nucleic acid molecule of the present invention comprising a nucleotide sequence encoding a polypeptide comprising a 3′-5′ exonuclease domain, or complement thereof. Preferably, the expression cassette comprises a nucleic acid molecule comprises a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37.

The present invention also relates to a vector comprising the nucleic acid molecules of the present invention encoding a polypeptide comprising a 3′-5′ exonuclease domain and/or activity. Preferably, the vector further comprises a promoter operably linked to the nucleic acid molecule of the present invention. More preferably, the vector further comprises a promoter and terminator, each operably linked to the nucleic acid molecule of the invention. Further, the present invention encompasses a plant cell (or a plant comprising such a plant cell) comprising a nucleic acid, recombinant nucleic acid molecule, an expression cassette or vector of the present invention encoding a polypeptide comprising a 3′-5′ exonuclease domain. The invention also provides progeny of the plant cells or plants described above, seeds, and parts of such a plant of the present invention, and the progeny thereof.

In yet a further aspect, the present invention also provides for methods to identify a compound that is capable of interacting with a polypeptide comprising a 3′-5′ exonuclease domain as described above. Preferably, the compound is capable of altering the activity of said polypeptide. The compound can alter the activity of the polypeptide by increasing or decreasing the polypeptide exonuclease or gene silencing activity. In a preferred embodiment, such compound is a nucleic acid molecule, such as an aptamer, or a small-molecule ligand. In another preferred embodiment, such compound is applied to a plant or a plant cell, and such application results in the alteration of the activity of a polypeptide comprising a 3′-5′ exonuclease domain in the plant or plant cell. Application of such a compound results in a more stable and predictable expression of a nucleotide sequence of interest in a plant cell or plant.

Thus, through an alteration of the expression of a nucleic acid molecule of the invention, the stable and predictable expression of a nucleic acid molecule of interest in a plant cell, the present invention provides a great advantage over current methods for the manipulation of gene expression in plant cells and plants. Current methods of transformation require extensive screening and testing of a large number of plants to identify a plant that stably and predictably expresses a nucleotide sequence of interest. Suppressing or decreasing expression of the nucleic acid molecule of the present invention results in decreased levels of post transcriptional gene silencing and improved expression of genes of interest. Therefore, the present invention allows for the production of improved plants, particularly improved commercial varieties, in a more timely and cost-effective manner.

The present invention thus provides:

An isolated nucleic acid molecule comprising a nucleotide sequence encoding a polypeptide comprising a 3′-5′ exonuclease domain, and wherein the polypeptide is identical or substantially similar to an amino acid sequence of SEQ ID NO:2, SEQ ID NO:4, SEQ ID NO:6, SEQ ID NO:22, SEQ ID NO:10, SEQ ID NO:12, SEQ ID NO:14, SEQ ID NO:16, SEQ ID NO:18, SEQ ID NO:24, SEQ ID NO:36 or SEQ ID NO:38 or complements thereof. Preferably, the polypeptide is identical or substantially similar to SEQ ID NO:2, SEQ ID NO:4, SEQ ID NO:6 or SEQ ID NO:22. More preferably, the polypeptide is identical or substantially similar to SEQ ID NO:2 or SEQ ID NO:24. In another preferred embodiment, the nucleotide sequence is identical or substantially similar to a nucleotide sequence of SEQ ID NO:1, SEQ ID NO:3, SEQ ID NO:5, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37. Preferably, the nucleotide sequence is identical or substantially similar to SEQ ID NO:1, SEQ ID NO:3, SEQ ID NO:5, SEQ ID NO:21, or SEQ ID NO:23. More preferably, the nucleotide sequence is substantially similar to SEQ ID NO:1. Most preferably, the nucleotide sequence is identical or substantially similar to SEQ ID NO:23. In another preferred embodiment, the 3′-5′ exonuclease domain preferably comprises an RNase D related domain Preferably, the polypeptide comprises 3′-5′ exonuclease activity, and most preferably, 3′-5′ RNA exonuclease activity. In yet another preferred embodiment, the nucleotide sequence is derived from a plant.

The present invention further provides an isolated recombinant nucleic acid molecule comprising a nucleotide sequence encoding a polypeptide encoded by the amino acid sequence identical or substantially similar to SEQ ID NO:2, SEQ ID NO:4, SEQ ID NO:6, SEQ ID NO:22, SEQ ID NO:10, SEQ ID NO:12, SEQ ID NO:14, SEQ ID NO:16, SEQ ID NO:18, SEQ ID NO:24, SEQ ID NO:36 or SEQ ID NO:38, or complements thereof. More preferably, the recombinant nucleic acid molecules comprise the nucleotide sequence of SEQ ID NO:23 or complement thereof. The recombinant nucleic acid molecule is operatively linked to a promoter functional in a cell. Preferably, the promoter is functional in a plant cell. Preferably, the nucleotide sequence of the present invention is in sense orientation in the nucleic acid molecule or in anti-sense orientation in the recombinant nucleic acid molecule. In yet another preferred embodiment, the polypeptide does not encode or comprise a helicase domain.

The present invention further provides:

An isolated and substantially purified polypeptide comprising an amino acid sequence identical or substantially similar to SEQ ID NO:2, SEQ ID NO:4, SEQ ID NO:6, SEQ ID NO:22, SEQ ID NO:10, SEQ ID NO:12, SEQ ID NO:14, SEQ ID NO:16 SEQ ID NO:18, SEQ ID NO:24, SEQ ID NO:36, or SEQ ID NO:38. Preferably, the polypeptide comprises the amino acid sequence of SEQ ID NO:24. Alternatively, the polypeptide consists of the amino acid sequence of SEQ ID NO:24.

The present invention further provides:

An expression cassette comprising a nucleic acid or DNA molecule of the present invention. Preferably, the expression cassette further comprises a promoter and terminator. More preferably, the promoter is a constitutive promoter, an inducible promoter, a tissue-specific promoter or a developmentally-regulated promoter.

A vector comprising the nucleic acid molecules of the present invention.

A cell comprising the nucleic acid or recombinant nucleic acid molecule of the present invention, and a cell comprising the expression cassette of the present invention Preferably, the cell is a plant cell. In a preferred embodiment, the nucleotide sequence of the present invention is expressed in said plant cell. In another preferred embodiment, the expression cassette promoter is a constitutive promoter, an inducible promoter, a tissue-specific promoter or a developmentally-regulated promoter. In another preferred embodiment, the expression cassette or recombinant nucleic acid molecule is stably integrated in the genome of the plant cell. In yet another preferred embodiment, the plant cell comprises an endogenous nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37. Preferably, the endogenous nucleotide sequence is identical or substantially similar to SEQ ID NO:1, SEQ ID NO:3, SEQ ID NO:5 SEQ ID NO:21, or SEQ ID NO:23. More preferably, the endogenous nucleotide sequence is identical or substantially similar to SEQ ID NO:1. Most preferably, the nucleotide sequence is identical or substantially similar to SEQ ID NO:23. Preferably, the expression of said endogenous nucleotide sequence in said plant cell is altered.

In a further preferred embodiment, the plant cell or plant comprises a nucleic acid molecule, or recombinant nucleic acid molecule, or expression cassette or vector of the present invention and further comprises a nucleic acid molecule comprising a nucleotide sequence of interest, wherein the expression of said nucleotide sequence of interest in said plant cell is altered as compared to the expression of said nucleotide sequence of interest in a plant cell lacking said nucleic acid molecule of the present invention. In another embodiment, the nucleotide sequence of interest is operably linked to a promoter. In yet another embodiment, the nucleotide sequence of interest is in an expression cassette.

The invention further provides a plant comprising the plant cell, and progeny and seeds from the plant comprising a nucleic acid sequence of the present invention.

The present invention further provides:

A plant cell comprising an endogenous nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 and wherein said plant cell comprises a mutation in said endogenous nucleotide sequence, or in a regulatory region thereof. Preferably, said mutation is due to the insertion of a nucleic acid molecule into said endogenous nucleotide sequence or into a regulatory region thereof, wherein the expression of said endogenous nucleotide sequence in said plant is altered. Preferably, the endogenous nucleotide sequence is identical or substantially similar to SEQ ID NO:1. Most preferably, the nucleotide sequence is as described or substantially similar to SEQ ID NO:23. Preferably, the insertion of a nucleic acid molecule comprises one T-DNA border region or a transposable element. An advantage of the invention is that the expression of said endogenous nucleotide sequence in said plant cell is reduced. In another preferred embodiment, the mutation is due to a deletion. In yet another embodiment, the mutation is due to a point mutation.

Preferably, the plant cell further comprises an expression cassette comprising a nucleotide sequence of interest, wherein the expression of said nucleotide sequence of interest in said plant cell is stabilized or increased as compared to the expression of said nucleotide sequence of interest in a plant cell lacking said nucleic acid molecule of the present invention. In another preferred embodiment, the expression of said endogenous nucleotide sequence described above in said plant cell is increased

In yet another preferred embodiment, plant cell further comprises an expression cassette comprising a nucleotide sequence of interest, wherein the expression of said nucleotide sequence of interest in said plant cell is decreased as compared to the expression of said nucleotide sequence of interest in a plant cell lacking said nucleic acid molecule of the present invention.

A plant comprising the plant cell comprising the above-described nucleic acid molecules or expression cassettes, or recombinant nucleic acid molecules.

The present invention further provides:

A plant cell or plant capable of expressing a sense RNA molecule of a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37 and an anti-sense RNA molecule of said nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37 wherein said sense and said anti-sense RNA molecules are capable of forming a double-stranded RNA molecule. An advantage of the invention is that the expression in said plant cell of an endogenous nucleotide sequence of said plant cell that is substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 is reduced.

In another preferred embodiment, the plant cell further comprises an expression cassette comprising a second nucleotide sequence, wherein the expression of said second nucleotide sequence in said plant cell is stabilized or increased as compared to the expression of said second nucleotide sequence in a plant cell that is not expressing said sense and said anti-sense RNA molecules.

A plant, seed or progeny thereof comprising the plant cell comprising the sense and antisense constructs as described above.

The present invention further provides:

A method for altering the expression of an endogenous nucleotide sequence that is identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 in a plant cell or plant comprising the step of: altering the transcription or translation of said endogenous nucleotide sequence in said plant cell or plant.

In a preferred embodiment, wherein altering the transcription or translation of said endogenous nucleotide sequence in said plant cell or plant comprises the step of:

a) expressing in said plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37 or a portion thereof, in sense orientation; or

b) expressing in said plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37 or a portion thereof, in anti-sense orientation; or

c) expressing in said plant cell a sense RNA of a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, and an anti-sense RNA of said nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, wherein said sense and said anti-sense RNAs are capable of forming a double-stranded RNA molecule; or

d) expressing in said plant cell a ribozyme capable of specifically cleaving a messenger RNA transcript encoded by a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37; or

e) modifying by homologous recombination in said plant cell at least one chromosomal copy of the nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or of a regulatory region thereof; or

f) expressing in said plant cell a zinc finger protein that is capable of binding to a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37, or to a regulatory region thereof; or

g) introducing into said plant cell a chimeric oligonucleotide that is capable of modifying at least one chromosomal copy of the nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or a regulatory region thereof.

The present invention further provides:

A method for altering the expression of an endogenous nucleotide sequence that is as described or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 in a plant cell comprising introducing into said plant cell a means for altering the transcription or translation of said endogenous nucleotide sequence in said plant cell.

The present invention further provides:

A method for altering the expression of a nucleotide sequence of interest in a plant cell or plant comprising the steps of:

a) altering the expression in said plant cell or plant of an endogenous nucleotide sequence of said plant cell that is identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37; and

b) introducing into said plant cell a nucleic acid molecule comprising said nucleotide sequence of interest, wherein the expression of said nucleotide sequence of interest in said plant cell or plant is altered.

In a preferred embodiment, said step a) comprises:

a) expressing in said plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, in sense orientation; or

b) expressing in said plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, in anti-sense orientation; or

c) expressing in said plant cell a sense RNA of a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, and an anti-sense RNA of said nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, wherein said sense and said anti-sense RNAs are capable of forming a double-stranded RNA molecule; or

d) expressing in said plant cell a ribozyme capable of specifically cleaving a messenger RNA transcript encoded by a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or

e) modifying by homologous recombination in said plant cell at least one chromosomal copy of the nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37, or of a regulatory region thereof; or

f) expressing in said plant cell a zinc finger protein that is capable of binding to a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37, or to a regulatory region thereof; or

g) introducing into said plant cell a chimeric oligonucleotide that is capable of modifying at least one chromosomal copy of the nucleotide identical or sequence substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37 or a regulatory region thereof.

The present invention further provides:

A method for altering, increasing or stabilizing the expression of a nucleotide sequence of interest in a plant cell comprising the steps of:

a) obtaining a plant cell comprising an expression cassette of the present invention expressing the nucleotide sequence of the present invention; and

b) introducing into said plant cell a nucleic acid molecule comprising said nucleotide sequence of interest, wherein the expression of said nucleotide sequence of interest in said plant cell is altered, increased or stabilized as compared to the expression of said nucleotide sequence of interest in a plant cell lacking said expression cassette.

Alternatively, the expression of said nucleotide sequence of interest in said plant cell is reduced or increased. Preferably, the nucleotide sequence of interest is identical or substantially similar to an endogenous nucleotide sequence of said plant cell.

The present invention further provides:

A method for stabilizing the expression of a nucleotide sequence of interest in a plant cell comprising:

a) altering the expression in a plant cell of an endogenous nucleotide sequence of said plant cell that encodes a polypeptide comprising a 3′-5′ exonuclease domain, and wherein said polypeptide is identical or substantially similar to an amino acid sequence selected from the group consisting of SEQ ID NO:2, SEQ ID NO:4, SEQ ID NO:6, SEQ ID NO:22, SEQ ID NO:10, SEQ ID NO:12, SEQ ID NO:14, SEQ ID NO:16, SEQ ID NO:18, SEQ ID NO:24, SEQ ID NO:36 and SEQ ID NO:38; and

b) introducing into said plant cell a nucleotide sequence of interest,

wherein the expression of said nucleotide sequence of interest in said plant cell is stabilized.

Preferably, the polypeptide has 3′-5′ RNA exonuclease activity. Preferably, the 3′-5′ exonuclease domain is an RNase D related domain. Preferably, the endogenous nucleotide sequence is identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37.

Preferably, the expression of said endogenous nucleotide sequence is altered by:

a) expressing in said plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, in sense orientation; or

b) expressing in said plant cell a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, in anti-sense orientation; or

c) expressing in said plant cell a sense RNA of a nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, and an anti-sense RNA of said nucleotide sequence identical or substantially similar to SEQ ID NO:1, SEQ ID NO:3, SEQ ID NO:5, SEQ ID NO:7, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37, or a portion thereof, wherein said sense and said anti-sense RNAs are capable of forming a double-stranded RNA molecule; or

d) expressing in said plant cell a ribozyme capable of specifically cleaving a messenger RNA transcript encoded by a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37; or

e) expressing in said plant cell an aptamer specifically directed to a polypeptide identical or substantially similar to SEQ ID NO:2, SEQ ID NO:4, SEQ ID NO:6, SEQ ID NO:22, SEQ ID NO:10, SEQ ID NO:12, SEQ ID NO:14, SEQ ID NO:24, SEQ ID NO:36 or SEQ ID NO:38; or

f) modifying by homologous recombination in said plant cell at least one chromosomal copy of the nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37, or of a regulatory region thereof; or

g) expressing in said plant cell a zinc finger protein that is capable of binding to a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37, or to a regulatory region thereof; or

h) introducing into said plant cell a chimeric oligonucleotide that is capable of modifying at least one chromosomal copy of the nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:23, SEQ ID NO:35 or SEQ ID NO:37, or a regulatory region thereof.

Preferably, the expression in a plant cell of said endogenous nucleotide sequence that encodes a polypeptide comprising a 3′-5′ exonuclease domain is reduced.

The present invention further provides:

A method for identifying a compound capable of interacting with a polypeptide comprising a 3′-5′ exonuclease domain comprising:

a) combining a polypeptide comprising the amino acid sequence set forth in SEQ ID NO:2, SEQ ID NO:4, SEQ ID NO:6, SEQ ID NO:22, SEQ ID NO:10, SEQ ID NO:12, SEQ ID NO:14, SEQ ID NO:16, SEQ ID NO:18, SEQ ID NO:24, SEQ ID NO:36 or SEQ ID NO:38, or a homolog thereof, and a compound to be tested for the ability to interact with said polypeptide, under conditions conducive to interaction; and

b) selecting a compound from step (a) that is capable of interacting with said polypeptide.

Preferably, the polypeptide is encoded by a nucleotide sequence identical or substantially similar to SEQ ID NO:1, SEQ ID NO:3, SEQ ID NO:5, SEQ ID NO:21, SEQ ID NO:9, SEQ ID NO:11, SEQ ID NO:13, SEQ ID NO:15, SEQ ID NO:17, SEQ ID NO:23, SEQ ID NO:35 or SEQ ID NO:37.

The present invention further provides:

A compound identifiable by the method disclosed immediately above. Preferably, the compound is capable of altering the activity of said polypeptide. More preferably, the compound is capable of decreasing or increasing gene silencing activity of the polypeptide.

### DESCRIPTION OF THE FIGURES

FIG. 1 is a schematic representation of the T-DNA region of plasmid p35S-GFP.

FIG. 2 is a schematic representation of the vector pRDP1.

### DESCRIPTION OF THE SEQUENCE LISTING

SEQ ID NO:1 nucleotide sequence corresponding to GenPept accession CAB36851
SEQ ID NO:2 GenPept accession CAB36851
SEQ ID NO:3 nucleotide sequence corresponding to GenPept accession AAD25623
SEQ ID NO:4 GenPept accession AAD25623
SEQ ID NO:5 nucleotide sequence corresponding to GenPept accession AAC69936
SEQ ID NO:6 GenPept accession AAC69936
SEQ ID NO:7 nucleotide sequence corresponding to GenPept accession AAC42241
SEQ ID NO:8 GenPept accession AAC42241
SEQ ID NO:9 nucleotide sequence corresponding to GenPept accession AAD26968
SEQ ID NO:10 GenPept accession AAD26968
SEQ ID NO:11 nucleotide sequence corresponding to GenPept accession AAC25931
SEQ ID NO:12 GenPept accession AAC25931
SEQ ID NO:13 nucleotide sequence corresponding to GenPept accession AAF98185
SEQ ID NO:14 GenPept accession AAF98185
SEQ ID NO:15 nucleotide sequence corresponding to GenPept accession CAA80137
SEQ ID NO:16 GenPept accession CAA80137
SEQ ID NO:17 nucleotide sequence corresponding to GenPept accession AAF06162
SEQ ID NO:18 GenPept accession AAF06162
SEQ ID NO:19 Oligonucleotide 3′ specific primer
SEQ ID NO:20 Oligonucleotide pD991 primer
SEQ ID NO:21 corrected nucleotide sequence corresponding to corrected GenPept accession AAC42241
SEQ ID NO:22 corrected GenPept accession AAC42241
SEQ ID NO:23 nucleotide sequence of cDNA encoding a polypeptide comprising a RNase D related domain from Arabidopsis thaliana
SEQ ID NO:24 amino acid sequence of polypeptide comprising a RNase D related domain from Arabidopsis thaliana
SEQ ID NO:25 oligonucleotide T-DNA specific primer LB1
SEQ ID NO:26 oligonucleotide T-DNA specific primer LB2
SEQ ID NO:27 oligonucleotide T-DNA specific primer LB3
SEQ ID NO:28 oligonucleotide arbitrary degenerate primer AD3
SEQ ID NO:29 oligonucleotide primer 36851TD#3
SEQ ID NO:30 gene-specific oligonucleotide primer L22F4F
SEQ ID NO:31 gene-specific oligonucleotide primer F22L4R
SEQ ID NO:32 oligonucleotide primer AtWRN CDS F
SEQ ID NO:33 oligonucleotide primer AtWRN-RT-R
SEQ ID NO:34 oligonucleotide primer AtWRN CDS R
SEQ ID NO:35 nucleotide sequence corresponding to GenPept accession AAG50917
SEQ ID NO:36 GenPept accession AAG50917
SEQ ID NO:37 nucleotide sequence corresponding to GenPept accession BAB11227
SEQ ID NO:38 GenPept accession BAB11227

### Definitions

For clarity, certain terms used in the specification are defined and used as follows:

Alter: to “alter” the expression of a nucleotide sequence in a plant cell means that the level of expression of the nucleotide sequence in a plant cell after applying a method of the present invention is different from its expression in the cell before applying the method. In a preferred embodiment, to alter expression means that the expression of the nucleotide sequence in the plant is reduced after applying a method of the present invention as compared to before applying the method. The term “Reduced” means herein lower, preferably significantly lower, more preferably the expression of the nucleotide sequence is not detectable. In another preferred embodiment, to alter expression means that the expression of the nucleotide sequence in the plant is increased after applying a method of the present invention as compared to before applying the method.

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Complementary: “Complementary” refers to two nucleotide sequences which comprise antiparallel nucleotide sequences capable of pairing with one another upon formation of hydrogen bonds between the complementary base residues in the antiparallel nucleotide sequences.

DNA shuffling: DNA shuffling is a method to rapidly, easily and efficiently introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA encodes an enzyme modified with respect to the enzyme encoded by the template DNA, and preferably has an altered biological activity with respect to the enzyme encoded by the template DNA.

Double-stranded RNA: A “double-stranded RNA (dsRNA)” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence which is present in the genome of the untransformed plant cell.

Essential: An “essential” gene is a gene encoding a protein such as e.g. a biosynthetic enzyme, receptor, signal transduction protein, structural gene product, or transport protein that is essential to the growth or survival of the plant.

Expression: “Expression” refers to the transcription and/or translation of a nucleotide sequence, for example an endogenous gene or a heterologous gene, in a cell. In the case of antisense constructs, for example, expression may refer to the transcription of the antisense DNA only.

Expression cassette: “Expression cassette” as used herein means a DNA sequence capable of directing expression of a particular nucleotide sequence in an appropriate host cell, comprising a promoter functional in the plant cell into which it will be introduced, operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue or organ or stage of development.

Heterologous DNA Sequence: a DNA sequence not naturally associated with a host cell into which it is introduced, including non-naturally occurring multiple copies of a endogenous DNA sequence.

Homologous DNA Sequence: a DNA sequence naturally associated with a host cell.

Isogenic: plants which are genetically identical, except that they may differ by the presence or absence of a heterologous DNA sequence.

Isolated: in the context of the present invention, an isolated DNA molecule or an isolated enzyme is a DNA molecule or enzyme which, by the hand of man, exists apart from its native environment and is therefore not a product of nature. An isolated DNA molecule or enzyme may exist in a purified form or may exist in a non-native environment such as, for example, in a transgenic host cell.

Mature protein: protein which is normally targeted to a cellular organelle, such as a chloroplast, and from which the transit peptide has been removed.

Minimal Promoter: promoter elements, particularly a TATA element, that are inactive or that have greatly reduced promoter activity in the absence of upstream activation. In the presence of a suitable transcription factor, the minimal promoter functions to permit transcription.

“Nucleic Acids” and “Nucleotides” refer to naturally occurring or synthetic or artificial nucleic acid or nucleotides

Plant: A “plant” refers to any plant or part of a plant at any stage of development. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units.

Pre-protein: protein which is normally targeted to a cellular organelle, such as a chloroplast, and still comprising its transit peptide.

Significant Increase or Decrease: an increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Stabilize: to “stabilize” the expression of a nucleotide sequence in a plant cell means that the level of expression of the nucleotide sequence after applying a method of the present invention is approximately the same in cells from the same tissue in different plants from the same generation or throughout multiple generations when the plants are grown under the same or comparable conditions.

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably, the substantially similar nucleotide sequence encodes the polypeptide encoded by the reference nucleotide sequence. The term “substantially similar” is specifically intended to include nucleotide sequences wherein the sequence has been modified to optimize expression in particular cells. The percentage of identity between the amino acid sequence encoded by the substantially similar nucleotide sequence and the reference nucleotide sequence is desirably at least 24%, more desirably at least 30%, more desirably at least 45%, preferably at least 60%, more preferably at least 75%, still more preferably at least 90%, yet still more preferably at least 95%, yet still more preferably at least 99%. Sequence comparisons are carried out using default GAP analysis with the University of Wisconsin GCG, SEQWEB application of GAP, based on the algorithm of Needleman and Wunsch (Needleman and Wunsch (1970) J. Mol. Biol. 48: 443-453). A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. Homologs of the nucleotide sequence include nucleotide sequences that encode an amino acid sequence that is at least 24% identical, more preferably at least 35% identical, yet more preferably at least 50% identical, yet more preferably at least 65% identical to the reference amino acid sequence, as measured using the parameters described above, wherein the amino acid sequence encoded by the homolog has the biological activity of a 3′-5′ exonuclease. More preferably, the homolog has the biological activity of a 3′-5′ RNA exonuclease. In another preferred embodiment, a homolog of the nucleotide sequence encodes an amino acid sequence that comprises a 3′-5′ exonuclease domain.

The term “substantially similar”, when used herein with respect to a polypeptide, means a protein corresponding to a reference polypeptide, wherein the polypeptide has substantially the same structure and function as the reference protein, e.g. where only changes in amino acids sequence not affecting the polypeptide function occur. When used for a polypeptide or an amino acid sequence the percentage of identity between the substantially similar and the reference polypeptide or amino acid sequence desirably is at least 24%, more desirably at least 30%, more desirably at least 45%, preferably at least 60%, more preferably at least 75%, still more preferably at least 90%, yet still more preferably at least 95%, yet still more preferably at least 99%, using default GAP analysis parameters as described above. Homologs are amino acid sequences that are at least 24% identical, more preferably at least 35% identical, yet more preferably at least 50% identical, yet more preferably at least 65% identical to the reference polypeptide or amino acid sequence, as measured using the parameters described above, wherein the amino acid sequence encoded by the homolog has the biological activity of a 3′-5′ exonuclease. More preferably, the homolog has the biological activity of a 3′-5′ RNA exonuclease. In another preferred embodiment, a homolog of the nucleotide sequence encodes an amino acid sequence that comprises a 3′-5′ exonuclease domain.

Target gene: A “target gene” is any gene in a plant cell. For example, a target gene is a gene of known function or is a gene whose function is unknown, but whose total or partial nucleotide sequence is known. Alternatively, the function of a target gene and its nucleotide sequence are both unknown. A target gene is a native gene of the plant cell or is a heterologous gene which has previously been introduced into the plant cell or a parent cell of said plant cell, for example by genetic transformation. A heterologous target gene is stably integrated in the genome of the plant cell or is present in the plant cell as an extrachromosomal molecule, e.g. as an autonomously replicating extrachromosomal molecule.

Transformation: a process for introducing heterologous nucleic acid molecule into a cell, tissue, or plant. Transformed cells, tissues, or plants are understood to encompass not only the end product of a transformation process, but also transgenic progeny thereof.

Transgenic: transformed, preferably stably transformed, with a recombinant DNA molecule that preferably comprises a suitable promoter operatively linked to a DNA sequence of interest.

### DETAILED DESCRIPTION OF THE INVENTION

The ability to reproducibly and predictably manipulate gene expression in plants is an important consideration for the production of novel commercial varieties with improved properties. New traits are often introduced into plant cells by transgenic methods but their expression is sometimes subject to variations between individual plants or between different generations. This phenomenon is referred to as gene silencing and the selection of lines not affected by gene silencing requires substantial efforts and is expensive. In other applications, it is desired to reduce or eliminate the expression of a particular endogenous gene in a plant cell, but with current methods it is often difficult to achieve this routinely in a stable and reproducible manner. Therefore, it is an object of the present invention to provide novel methods that address these needs and allow stabilizing or altering the expression of a nucleotide sequence of interest in a plant cell in a predictable and stable manner. According to the present invention, this is preferably achieved by altering the expression in a plant cell of a nucleotide sequence encoding a polypeptide having 3′-5′ exonuclease domain.

I. Nucleotide Sequences Encoding a Polypeptide Comprising a 3′-5′ Exonuclease Domain

In one aspect, the present invention provides for nucleic acid molecules having nucleotide sequence encoding polypeptides comprising a 3′-5′ exonuclease domain. Preferably, the 3′-5′ exonuclease domain is a RNase D related domain. The present invention also provides nucleic acid molecules comprising a nucleotide sequence encoding a polypeptide comprising 3′-5′ exonuclease activity. Preferably, the polypeptide has 3′-5′ RNA exonuclease activity. In yet another preferred embodiment, the nucleotide sequence is isolated from a plant, preferably from a monocotyledonous plant or a dicotyledonous plant. Preferably, the plants are, but not limited to, corn, rice, wheat, soybean, cotton, sunflower, Brassica spp., canola, tomato, potato, Solanaceae spp. or sugar beets. More preferably, the nucleic acid molecules are isolated from Arabidopsis thaliana.

A 3′-5′ exonuclease domain typically comprises three subdomains designated as exo I, exo II and exo III (Moser et al. (1997) Nucl. Acids Res. 25:5110-5118, incorporated herein by reference in its entirety). These motifs are clustered around the active site and contain four negatively charged residues that serve as ligands for the two metal ions necessary for catalysis in addition to a catalytically active tyrosine. Typically, a 3′-5′ exonuclease domain is approximately 140 amino acids long. 3′-5′ exonuclease domains are for example found in DNA polymerases where they are sometimes referred to as the 3′-5′ exodeoxyribonuclease (or proofreading) domains.

3′-5′ exonuclease domains are also found in the RNase D family of polypeptides, that includes for example the E. coli ribonuclease (RNase D), the S. cerevisiae Rrp6p protein and the human Werner syndrome protein (see Mian (1997) Nucleic Acids Research 25:3187-3195, incorporated herein by reference in its entirety). Such domains are referred to as RNase D related domains. An alignment of polypeptides comprising an RNase D related domain is shown in Mian (1997). RNase D related domains and proofreading domains appear to be similar.

The inventors of the present invention are the first to screen for plant nucleotide sequences encoding a polypeptide comprising a 3′-5′ exonuclease domain, and to successfully identify such nucleotide sequences. This is carried out according to the methods disclosed in Example 1. The amino acid sequences and corresponding nucleotide sequences identified using the method and algorithms disclosed in Example 1 are set forth in SEQ ID NO:1-14, and 35-38 and briefly described as follows. An amino acid sequence predicted from a genomic sequence from Arabidopsis thaliana is found in GenBank under accession #CAB36851 and is set forth in SEQ ID NO:2. The corresponding nucleotide sequence is found in BAC F18A5, GenBank accession number AL035528.2. An amino acid sequence predicted from a genomic sequence from Arabidopsis thaliana is found in GenBank under accession #AAD25623 and is set forth in SEQ ID NO:4. The corresponding nucleotide sequence is found in BAC F20D21, GenBank accession number AC005287.4. An amino acid sequence predicted from a genomic sequence from Arabidopsis thaliana is found in GenBank under accession #AAC69936 and is set forth in SEQ ID NO:6. The corresponding nucleotide sequence is found in Arabidopsis thaliana chromosome II section 181 of 255, GenBank accession number AC005700.2. An amino acid sequence predicted from a genomic sequence from Arabidopsis thaliana is found in GenBank under accession #AAC42241 and is set forth in SEQ ID NO:8. The corresponding nucleotide sequence is found in Arabidopsis thaliana chromosome II section 145 of 255, GenBank accession number AC005395.2. An amino acid sequence predicted from a genomic sequence from Arabidopsis thaliana is found in GenBank under accession #AAD26968 and is set forth in SEQ ID NO:10. The corresponding nucleotide sequence is found in Arabidopsis thaliana chromosome II section 197 of 255, GenBank accession number AC007135.7. An amino acid sequence predicted from a genomic sequence from Arabidopsis thaliana is found in GenBank under accession #AAC25931 and is set forth in SEQ ID NO:12. The corresponding nucleotide sequence is found in Arabidopsis thaliana chromosome II section 182 of 255, GenBank accession number AC004681.2. An amino acid sequence predicted from a genomic sequence from Arabidopsis thaliana is found in GenBank under accession #AAF98185 and is set forth in SEQ ID NO:14. The corresponding nucleotide sequence is found in BAC F17F8, GenBank accession number AC000107.2. An amino acid sequence predicted from a genomic sequence from Arabidopsis thaliana is found in GenBank under accession # AAG50917 and is set forth in SEQ ID NO:36. The corresponding nucleotide sequence is found in BAC F14G9, GenBank accession number AC069159. An amino acid sequence predicted from a genomic sequence from Arabidopsis thaliana is found in GenBank under accession # BAB11227 and is set forth in SEQ ID NO:38. The corresponding nucleotide sequence is found in BAC K16H17, GenBank accession number AB016884.

The inventors of the present invention also discovered that the 5′ end of GenPept accession AAC42241 is missing due to incorrect annotation, and that GenPept accession AAC42241 lacks the exo I motif of the 3′-5′ exonuclease domain. The amino acid sequence comprising the entire 3′-5′ exonuclease domain (including exo 1) is disclosed for the first time in the instant application and is set forth in SEQ ID NO:22. The corresponding nucleotide sequence is set forth in SEQ ID NO:21.

Further, the present invention provides for nucleic acid molecules encoding a full length nucleotide sequence encoding a polypeptide comprising a 3′-5′ exonuclease domain of SEQ ID NO:24 as was cloned from Arabidopsis thaliana as set forth in Examples 2-3. The invention also provides a nucleic acid molecule comprising or having the sequence identical or substantially similar to the nucleotide sequence of SEQ ID NO:23 or complements thereof. The inventors of the present invention predicted a 3′-5′ exonuclease domain between about amino acid positions 129 and 287 in the amino acid sequence set forth in SEQ ID NO:2. The inventors of the present invention also predicted that the amino acid sequence between about amino acid positions 136 and 271 in SEQ ID NO:4 is comprised in a 3′-5′ exonuclease domain, that the amino acid sequence between about amino acid positions 76 and 210 in SEQ ID NO:6 is comprised in a 3′-5′ exonuclease domain, that the amino acid sequence between about amino acid positions 46 and 199 in SEQ ID NO:22 is comprised in a 3′-5′ exonuclease domain, that the amino acid sequence between about amino acid positions 57 and 193 in SEQ ID NO:10 is comprised in a 3′-5′ exonuclease domain, that the amino acid sequence between about amino acid positions 66 and 202 in SEQ ID NO:12 is comprised in a 3′-5′ exonuclease domain, that the amino acid sequence between about amino acid positions 380 and 538 in SEQ ID NO:36 is comprised in a 3′-5′ exonuclease domain, that the amino acid sequence between about amino acid positions 30 and 193 in SEQ ID NO:38 is comprised in a 3′-5′ exonuclease domain. The inventors of the present invention also predict that the amino acid sequence between about amino acid positions 129 and 282 in SEQ ID NO:24 comprises a 3′-5′ exonuclease domain.

Preferably, the nucleotide sequence of the present invention encode a polypeptide comprising a 3′-5′ exonuclease domain. In another aspect of the invention, the nucleotide sequence encodes a polypeptide comprising at least one 3′-5′ exonuclease domain. In yet another embodiment, the nucleotide sequence encodes a polypeptide comprising more than one 3′-5′ exonuclease domain.

Thus, the present invention discloses a nucleotide sequence encoding a polypeptide identical or substantially similar to SEQ ID NO:2, SEQ ID NO:4, SEQ ID NO:6, SEQ ID NO:22, SEQ ID NO:10, SEQ ID NO:12, SEQ ID NO:14, SEQ ID NO:36 or SEQ ID NO:38. Preferably, the polypeptide is identical or substantially similar to SEQ ID NO:2, SEQ ID NO:4, SEQ ID NO:6, or SEQ ID NO:22. More preferably, the polypeptide is identical or substantially similar to SEQ ID NO:2. Most preferably, the polypeptide is identical or substantially similar to the amino acid sequence of SEQ ID NO:24.

Preferably, the nucleotide sequence is identical or substantially similar to SEQ ID NO:1, SEQ ID NO:3, SEQ ID NO:5, SEQ ID NO:21, SEQ ID NO:9, SEQ ID NO:11 SEQ ID NO:13, SEQ ID NO:23, SEQ ID NO:35 or SEQ ID NO:37. More preferably, the nucleotide sequence is substantially similar to SEQ ID NO:1, SEQ ID NO:3, SEQ ID NO:5, or SEQ ID NO:21. Yet more preferably, the nucleotide sequence is identical or substantially similar to SEQ ID NO:1. Most preferably, the nucleotide sequence is identical or substantially similar to SEQ ID NO:23.

The inventors of the present invention are also the first to predict and demonstrate that a nucleotide sequence of the present invention is involved in gene silencing, and to use such nucleotide sequences to alter or stabilize the expression of a nucleotide sequence of interest in a cell as set forth in Example 5. The nucleotide sequences of the present invention are useful to alter or stabilize the expression of another nucleotide sequence of interest in a plant cell.

Based on Applicants' disclosure of the present invention, nucleotide sequences encoding polypeptides identical or substantially similar to SEQ ID NO:2, SEQ ID NO:4, SEQ ID NO:6, SEQ ID NO:8, SEQ ID NO:10, SEQ ID NO:12, SEQ ID NO:14, SEQ ID NO:24, SEQ ID NO:36 or SEQ ID NO:38 are isolated, preferably from the genome of any desired plant. For example, all or part of the nucleotide sequence set forth in SEQ ID NO:1 is used as a probe that selectively hybridizes to other nucleotide sequences present in a population of cloned genomic DNA fragments or cDNA fragments (i.e. genomic or cDNA libraries) from a chosen source organism. Such techniques include hybridization screening of plated DNA libraries (either plaques or colonies; see, e.g. Sambrook et al., “Molecular Cloning”, eds., Cold Spring Harbor Laboratory Press. (1989)) and amplification by PCR using oligonucleotide primers corresponding to sequence domains conserved among such polypeptides (see, e.g. Innis et al., “PCR Protocols, a Guide to Methods and Applications”, Academic Press (1990)). For example, oligonucleotide primers corresponding to a portion of a 3′-5′ exonuclease domain are used. These methods are particularly well suited to the isolation of nucleotide sequences from organisms closely related to the organism from which the probe sequence is derived. Isolation of such a nucleic acid molecule of the present invention, in particular SEQ ID NO:23, is described in Example 7.

The isolated nucleotide sequences taught by the present invention are manipulated according to standard genetic engineering techniques to suit any desired purpose. For example, they may be used as a probe capable of specifically hybridizing to coding sequences and messenger RNAs. To achieve specific hybridization under a variety of conditions, such probes include preferably at least 10 nucleotides in length, preferably at least 20 nucleotides in length, and most preferably at least 50 nucleotides in length. Such probes are used to amplify and analyze nucleotide sequences from a chosen organism via PCR.

Specific hybridization probes also are used to map the location of these native genes in the genome of a chosen plant using standard techniques based on the selective hybridization of the probe to genomic sequences. These techniques include, but are not limited to, identification of DNA polymorphisms identified or contained within the probe sequence, and use of such polymorphisms to follow segregation of the gene relative to other markers of known map position in a mapping population derived from self fertilization of a hybrid of two polymorphic parental lines (see e.g. Helentjaris et al., Plant Mol. Biol. 5: 109 (1985); Sommer et al. BioTechniques 12:82 (1992); D'Ovidio et al., Plant Mol. Biol. 15:169 (1990)). Mapping of genes in this manner is contemplated to be particularly useful for breeding purposes. For instance, by knowing the genetic map position of a mutant gene, flanking DNA markers are identified from a reference genetic map (see, e.g., Helentjaris, Trends Genet. 3: 217 (1987)). During introgression of the herbicide resistance trait into a new breeding line, these markers are used to monitor the extent of linked flanking chromosomal DNA still present in the recurrent parent after each round of back-crossing. Specific hybridization probes also are used to quantify levels of mRNA in a plant using standard techniques such as Northern blot analysis.

In another aspect of the present invention, a nucleotide sequence encoding a polypeptide comprising a 3′-5′ exonuclease domain is inserted in a recombinant nucleic acid molecule. The recombinant nucleic acid molecule is preferably operatively linked to a promoter. More preferably, the promoter is functional in a plant cell Recombinant nucleic acid molecules can be introduced into plant cells by genetic transformation, as described for example in further detail infra.

The present invention also provides for expression cassettes comprising a promoter operably linked to a nucleic acid molecule encoding polypeptides comprising 3′-5′ exonuclease domains described above and a terminator. The expression cassettes of the present invention may further comprise an enhancer.

In another aspect, the present invention provides vectors comprising the nucleic acid molecules encoding polypeptides comprising 3′-5′ exonuclease domains described above. Also, the vectors further comprise a promoter and terminator operationally linked to the nucleic acid molecule of the present invention. Plasmid and viral vectors known to those skilled in the art of molecular biology further comprising the nucleic acid molecules of the present invention are encompassed by the invention.

II. Methods for Altering the Expression of a Polypeptide Having 3′-5′ Exonuclease Domain in a Cell

The inventors of the present invention are the first to discover that nucleotide sequences of the present invention are useful to manipulate or alter gene expression or post-transcriptional gene silencing (PTGS). Preferably, gene expression or PTGS is manipulated or altered in plant cells. Thus, one object of the present invention is to alter the expression in a plant cell of a nucleotide sequence of said plant said that encodes a polypeptide comprising a 3′-5′ exonuclease domain and/or activity.

As described in Examples 5 and 6, decreasing or preventing expression of a nucleic acid molecule encoding a polypeptide comprising a 3′-5′ exonuclease domain, causes a decrease or eliminates detectable levels of PTGS. The levels of PTGS are determined by measuring the levels of expression of a GFP reporter gene. Replacement of such a sequence of the present invention, restores PTGS activity in the plant.

Additionally, overexpression of a nucleic acid molecule of the present invention encoding a polypeptide comprising a 3′-5′ exonuclease domain increases or supplements levels of PTGS.

The present invention provides a number of methods for altering the expression of a nucleic acid molecule encoding a polypeptide comprising a 3′-5′ exonuclease domain. These methods allow for the decrease or increase in the level of expression of the nucleic acid molecule encoding polypeptide comprising a 3′-5′ exonuclease domain which in turn, produces alteration of expression of nucleic acid molecules or genes of interest.

For example, the alteration in expression of the nucleic acid molecule of the present invention is achieved in one of the following ways:

(1) “Sense” Suppression

Alteration of the expression of a nucleotide sequence of the present invention, preferably reduction of its expression, is obtained by “sense” suppression (referenced in e.g. Jorgensen et al. (1996) Plant Mol. Biol. 31, 957-973). In this case, the entirety or a portion of a nucleotide sequence of the present invention is comprised in a DNA molecule. The DNA molecule is preferably operatively linked to a promoter functional in a cell comprising the target gene, preferably a plant cell, and introduced into the cell, in which the nucleotide sequence is expressible. The nucleotide sequence is inserted in the DNA molecule in the “sense orientation”, meaning that the coding strand of the nucleotide sequence can be transcribed. In a preferred embodiment, the nucleotide sequence is fully translatable and all the genetic information comprised in the nucleotide sequence, or portion thereof, is translated into a polypeptide. In another preferred embodiment, the nucleotide sequence is partially translatable and a short peptide is translated. In a preferred embodiment, this is achieved by inserting at least one premature stop codon in the nucleotide sequence, which bring translation to a halt. In another more preferred embodiment, the nucleotide sequence is transcribed but no translation product is being made. This is usually achieved by removing the start codon, e.g. the “ATG”, of the polypeptide encoded by the nucleotide sequence. In a further preferred embodiment, the DNA molecule comprising the nucleotide sequence, or a portion thereof, is stably integrated in the genome of the plant cell. In another preferred embodiment, the DNA molecule comprising the nucleotide sequence, or a portion thereof, is comprised in an extrachromosomally replicating molecule.

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

(2) “Anti-Sense” Suppression

In another preferred embodiment, the alteration of the expression of a nucleotide sequence of the present invention, preferably the reduction of its expression is obtained by “anti-sense” suppression. The entirety or a portion of a nucleotide sequence of the present invention is comprised in a DNA molecule. The DNA molecule is preferably operatively linked to a promoter functional in a plant cell, and introduced in a plant cell, in which the nucleotide sequence is expressible. The nucleotide sequence is inserted in the DNA molecule in the “anti-sense orientation”, meaning that the reverse complement (also called sometimes non-coding strand) of the nucleotide sequence can be transcribed. In a preferred embodiment, the DNA molecule comprising the nucleotide sequence, or a portion thereof, is stably integrated in the genome of the plant cell. In another preferred embodiment the DNA molecule comprising the nucleotide sequence, or a portion thereof, is comprised in an extrachromosomally replicating molecule. Several publications describing this approach are cited for further illustration (Green, P. J. et al., Ann. Rev. Biochem. 55:569-597 (1986); van der Krol, A. R. et al, Antisense Nuc. Acids & Proteins, pp. 125-141 (1991); Abel, P. P. et al., Proc. Natl. Acad. Sci. USA 86:6949-6952 (1989); Ecker, J. R. et al., Proc. Natl. Acad. Sci. USA 83:5372-5376 (August 1986)).

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

(3) Homologous Recombination

In another preferred embodiment, at least one genomic copy corresponding to a nucleotide sequence of the present invention is modified in the genome of the plant by homologous recombination as further illustrated in Paszkowski et al., EMBO Journal 7:4021-26 (1988). This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence of the present invention are modified. Such regulatory elements are easily obtainable by screening a genomic library using the nucleotide sequence of the present invention, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also contemplated in the present invention. More recent refinements of this technique to disrupt endogenous plant genes have been described (Kempin et al., Nature 389:802-803 (1997) and Miao and Lam, Plant J., 7:359-365 (1995).

In another preferred embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the present invention and to contain the desired nucleotide change. For example, this technique is further illustrated in U.S. Pat. No. 5,501,967 and Zhu et al. (1999) Proc. Natl. Acad. Sci. USA 96: 8768-8773.

(4) Ribozymes

In a further embodiment, the RNA coding for a polypeptide of the present invention is cleaved by a catalytic RNA, or ribozyme, specific for such RNA. The ribozyme is expressed in transgenic plants and results in reduced amounts of RNA coding for the polypeptide of the present invention in plant cells, thus leading to reduced amounts of polypeptide accumulated in the cells. This method is further illustrated in U.S. Pat. No. 4,987,071.

(5) Dominant-Negative Mutants

In another preferred embodiment, the activity of the polypeptide encoded by the nucleotide sequences of this invention is changed. This is achieved by expression of dominant negative mutants of the proteins in transgenic plants, leading to the loss of activity of the endogenous protein.

(6) Aptamers

In a further embodiment, the activity of polypeptide of the present invention is inhibited by expressing in transgenic plants nucleic acid ligands, so-called aptamers, which specifically bind to the protein. Aptamers are preferentially obtained by the SELEX (Systematic Evolution of Ligands by EXponential Enrichment) method. In the SELEX method, a candidate mixture of single stranded nucleic acids having regions of randomized sequence is contacted with the protein and those nucleic acids having an increased affinity to the target are partitioned from the remainder of the candidate mixture. The partitioned nucleic acids are amplified to yield a ligand enriched mixture. After several iterations a nucleic acid with optimal affinity to the polypeptide is obtained and is used for expression in transgenic plants. This method is further illustrated in U.S. Pat. No. 5,270,163.

(7) Zinc Finger Proteins

A zinc finger protein that binds a nucleotide sequence of the present invention or to its regulatory region is also used to alter expression of the nucleotide sequence. Preferably, transcription of the nucleotide sequence is reduced or increased. Zinc finger proteins are for example described in Beerli et al. (1998) PNAS 95:14628-14633, or in WO 95/19431, WO 98/54311, or WO 96/06166, all incorporated herein by reference in their entirety.

(8) dsRNA

Alteration of the expression of a nucleotide sequence of the present invention is also obtained by dsRNA interference as described for example in WO 99/32619, WO 99/53050 or WO 99/61631, all incorporated herein by reference in their entirety.

(9) Insertion of a DNA Molecule (Insertional Mutagenesis)

In another preferred embodiment, a DNA molecule is inserted into a chromosomal copy of a nucleotide sequence of the present invention, or into a regulatory region thereof. Preferably, such DNA molecule comprises a transposable element capable of transposition in a plant cell, such as e.g. Ac/Ds, Em/Spm, mutator. Alternatively, the DNA molecule comprises a T-DNA border of an Agrobacterium T-DNA. The DNA molecule may also comprise a recombinase or integrase recognition site which can be used to remove part of the DNA molecule from the chromosome of the plant cell. An example of this method is set forth in Example 2. Methods of insertional mutagenesis using T-DNA, transposons, oligonucleotides or other methods known to those skilled in the art are also encompassed. Methods of using T-DNA and transposon for insertional mutagenesis are described in Winkler et al. (1989) Methods Mol. Biol. 82:129-136 and Martienssen (1998) PNAS 95:2021-2026, incorporated herein by reference in their entireties.

(10) Deletion Mutagenesis

In yet another embodiment, a mutation of a nucleic acid molecule of the present invention is created in the genomic copy of the sequence in the cell or plant by deletion of a portion of the nucleotide sequence or regulator sequence. Methods of deletion mutagenesis are known to those skilled in the art. See, for example, Miao et al, (1995) Plant J. 7:359. In yet another embodiment, this deletion is created at random in a large population of plants by chemical mutagenesis or irradiation and a plant with a deletion in a gene of the present invention is isolated by forward or reverse genetics. Irradiation with fast neutrons or gamma rays is known to cause deletion mutations in plants (Silverstone et al, (1998) Plant Cell, 10:155-169; Bruggemann et al., (1996) Plant J., 10:755-760; Redei and Koncz in Methods in Arabidopsis Research, World Scientific Press (1992), pp. 16-82). Deletion mutations in a gene of the present invention can be recovered in a reverse genetics strategy using PCR with pooled sets of genomic DNAs as has been shown in C. elegans (Liu et al., (1999), Genome Research, 9:859-867.). A forward genetics strategy would involve mutagenesis of a line displaying PTGS followed by screening the M2 progeny for the absence of PTGS. Among these mutants would be expected to be some that disrupt a gene of the present invention. This could be assessed by Southern blot or PCR for a gene of the present invention with genomic DNA from these mutants.

(11) Overexpression in a Plant Cell

In yet another preferred embodiment, a nucleotide sequence of the present invention encoding a polypeptide comprising a 3′-5′ exonuclease domain and/or activity in a plant cell is overexpressed. Examples of nucleic acid molecules and expression cassettes for overexpression of a nucleic acid molecule of the present invention are described infra (see Examples 8-10). Methods known to those skilled in the art of over-expression of nucleic acid molecules are also encompassed by the present invention.

In a preferred embodiment, the expression of the nucleotide sequence of the present invention is altered in every cell of a plant. This is for example obtained though homologous recombination or by insertion in the chromosome. This is also for example obtained by expressing a sense or antisense RNA, zinc finger protein or ribozyme under the control of a promoter capable of expressing the sense or antisense RNA, zinc finger protein or ribozyme in every cell of a plant. Constitutive expression, inducible, tissue-specific or developmentally-regulated expression are also within the scope of the present invention and result in a constitutive, inducible, tissue-specific or developmentally-regulated alteration of the expression of a nucleotide sequence of the present invention in the plant cell. Constructs for expression of the sense or antisense RNA, zinc finger protein or ribozyme, or for overexpression of a nucleotide sequence of the present invention, are prepared and transformed into a plant cell according to the teachings of the present invention, e.g. as described infra.

III. Methods for Manipulating the Expression of a Nucleotide Sequence of Interest in a Plant Cell

In another aspect of the present invention, a plant cell with altered expression of a nucleotide sequence of the present invention and as described above is used to alter or stabilize the expression of a nucleotide sequence of interest in a plant cell.

In a preferred embodiment, manipulation of the expression of a heterologous nucleotide sequence of interest is desired. In this case, the heterologous nucleotide sequence is introduced into an expression cassette. The heterologous nucleotide sequence is preferably introduced into a plant cell with altered expression of a nucleotide sequence encoding a polypeptide comprising a 3′-5′ exonuclease domain and/or activity. In a preferred embodiment, a plant cell with reduced expression of a nucleotide sequence encoding a polypeptide comprising a 3′-5′ exonuclease domain and/or activity is used to stabilize or to increase the expression of the nucleotide sequence of interest. Alternatively, a plant cell with increased expression of a nucleotide sequence encoding a polypeptide comprising a 3′-5′ exonuclease domain and/or activity is preferably used to reduce the expression of the nucleotide sequence of interest. Constitutive, inducible, tissue-specific or developmentally-regulated alteration of the nucleotide sequence of interest is preferably obtained by using a plant cell with constitutive, inducible, tissue-specific or developmentally-regulated alteration of the expression of the nucleotide sequence encoding a polypeptide comprising a 3′-5′ exonuclease domain.

In another preferred embodiment, the expression of an endogenous nucleotide sequence in a plant cell is manipulated using the present invention. In this case, a nucleotide sequence identical or substantially similar to the endogenous nucleotide sequence, or a reverse complement thereof, is introduced into a plant cell with altered expression of a nucleotide sequence of the present invention. In a preferred embodiment, a plant cell with increased expression of a nucleotide sequence of the present invention is preferably used to reduce the expression of the endogenous nucleotide sequence of interest.

Alternatively, a plant cell with reduced expression of a nucleotide sequence of the present invention is used to increase the expression of the nucleotide sequence of interest. Constitutive, inducible, tissue-specific or developmentally-regulated alteration of the endogenous nucleotide sequence is preferably obtained by using a plant cell with constitutive, inducible, tissue-specific or developmentally-regulated alteration of the expression of its nucleotide sequence encoding a polypeptide comprising a 3′-5′ exonuclease domain. Any portion of the endogenous nucleotide sequence is used. For example, if the nucleotide sequence comprises a coding region, the entire coding region or a portion thereof is used. Alternatively, a portion of the regulatory regions is used, preferably a transcribed portion of the regulatory region. Such portion is introduced into a recombinant nucleic acid molecule which is preferably introduced into an expression cassette or vector and transformed into a plant cell with altered expression of a nucleotide sequence of the present invention. Preferably, a nucleotide sequence used at least 70% identical to the endogenous nucleotide sequence, more preferably it is at least 80% identical, yet more preferably at least 90% identical, yet more preferably at least 95% identical, yet more preferably at least 99% identical.

A heterologous nucleotide sequence encodes for example, but not limited to, a polypeptide involved in waxy starch, herbicide tolerance, resistance for bacterial, fungal, or viral disease, insect resistance, enhanced nutritional quality, improved performance in an industrial process, altered reproductive capability, such as male sterility or male fertility, yield stability and yield enhancement. Using the present invention, such traits are stably and reproducibly expressed in a plant cell. Examples of endogenous nucleotide sequences of interest whose expression in a plant cell is altered using the present invention are found for example in WO 99/53050.

In another preferred embodiment, the nucleotide sequence of interest is derived from a pathogen of a plant, preferably a viral pathogen. Therefore, it is a further aspect of the present invention to provide for methods to control a pathogen. Preferably, a plant cell with altered expression of a nucleotide sequence that encodes a polypeptide comprising a 3′-5′ exonuclease domain is obtained as described above. Preferably, the plant cell further comprises a nucleotide sequence substantially similar to a nucleotide sequence derived from the pathogen. Preferably, increased expression of the nucleotide sequence that encodes a polypeptide comprising a 3′-5′ exonuclease domain results in increased gene silencing in the plant cell and increased resistance or tolerance to the pathogen.

III. Plant Transformation Technology

Nucleotide sequences of the present invention can be incorporated in plant or bacterial cells using conventional recombinant DNA technology. Generally, this involves inserting a nucleotide sequence of the present invention into an expression system to which the nucleotide sequence is heterologous (i.e., not normally present) using standard cloning procedures known in the art. The vector contains the necessary elements for the transcription and translation of the inserted protein-coding sequences in a host cell containing the vector. A large number of vector systems known in the art can be used, such as plasmids, bacteriophage viruses and other modified viruses. The components of the expression system optionally are modified to increase expression. For example, truncated sequences, nucleotide substitutions or other modifications optionally are employed. Expression systems known in the art are used to transform virtually any crop plant cell under suitable conditions. Transformed cells are regenerated into whole plants.

A. Requirements for Construction of Plant Expression Cassettes

Gene sequences intended for expression in transgenic plants are first operatively linked to a suitable promoter expressible in plants. Such expression cassettes optionally comprise further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes are easily transferred to the plant transformation vectors described infra. The following is a description of various components of typical expression cassettes.

1. Promoters

The selection of the promoter used determines the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters known in the art can be used. For example, for constitutive expression, the CaMV 35S promoter, the rice actin promoter, or the ubiquitin promoter may be used. For regulatable expression, the chemically inducible PR-1 promoter from tobacco or Arabidopsis may be used (see, e.g., U.S. Pat. No. 5,689,044).

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons.

3. Sequences for the Enhancement or Regulation of Expression

Numerous sequences are known to enhance gene expression from within the transcriptional unit and these sequences can be used in conjunction with the genes of this invention to increase their expression in transgenic plants. For example, various intron sequences such as introns of the maize AdhI gene have been shown to enhance expression, particularly in monocotyledonous cells. In addition, a number of non-translated leader sequences derived from viruses also are known to enhance expression, and these are particularly effective in dicotyledonous cells.

4. Coding Sequence Optimization

The coding sequence of the selected gene optionally is genetically engineered by altering the coding sequence for optimal expression in the crop species of interest. Methods for modifying coding sequences to achieve optimal expression in a particular crop species are well known (see, e.g. Perlak et al., Proc. Nat. Acad. Sci. USA 88: 3324 (1991); and Koziel et al., Bio/technol. 11: 194 (1993); Fennoy and Bailey-Serres. Nucl. Acids Res. 21: 5294-5300 (1993). Methods for modifying coding sequences by taking into account codon usage in plant genes and in higher plants, green algae, and cyanobacteria are well known (see table 4 in: Murray et al. Nucl. Acids Res. 17: 477-498 (1989); Campbell and Gowri Plant Physiol. 92: 1-11 (1990).

5. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). Other gene products are localized to other organelles such as the mitochondrion and the peroxisome (e.g. Unger et al. Plant Molec. Biol. 13: 411-418 (1989)). The cDNAs encoding these products are manipulated to effect the targeting of heterologous gene products to these organelles. In addition, sequences have been characterized which cause the targeting of gene products to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)). By the fusion of the appropriate targeting sequences described above to transgene sequences of interest one skilled in the art is able to direct the transgene product to any organelle or cell compartment.

B. Construction of Plant Transformation Vectors

Numerous transformation vectors available for plant transformation are known to those of ordinary skill in the plant transformation arts, and the genes pertinent to this invention are used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methotrexate (Bourouis et al., EMBO J. 2(7): 1099-1104 (1983)), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Typical vectors suitable for Agrobacterium transformation include the binary vectors pCIB200 and pCIB2001, as well as the binary vector pCIB10 and hygromycin selection derivatives thereof. (See, for example, U.S. Pat. No. 5,639,949).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Typical vectors suitable for non-Agrobacterium transformation include pCIB3064, pSOG19, and pSOG35. (See, for example, U.S. Pat. No. 5,639,949).

C. Transformation Techniques

Once the coding sequence of interest has been cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells.

Although a nucleotide sequence of the present invention can be inserted into any plant cell falling within these broad classes, it is particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane. Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. In each case the transformed cells are regenerated to whole plants using standard techniques known in the art. Transformation of most monocotyledon species has now also become routine. Preferred techniques include direct gene transfer into protoplasts using PEG or electroporation techniques, particle bombardment into callus tissue, as well as Agrobacterium-mediated transformation.

D. Plastid Transformation

In another preferred embodiment, a nucleotide sequence of the present invention is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit expression levels that can readily exceed 10% of the total soluble plant protein. In a preferred embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is for example extensively described in U.S. Pat. Nos. 5,451,513, 5,545,817, 5,545,818, and 5,877,462 in PCT application no. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps 12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Other selectable markers useful for plastid transformation are known in the art and encompassed within the scope of the invention.

IV. Recombinant Production of Polypeptides and Uses Thereof

In a further aspect, the present invention discloses the use of a nucleotide sequence of the present invention to recombinantly produce a polypeptide having 3′-5′ exonuclease activity. For recombinant production of a polypeptide in a host organism, a nucleotide sequence of the present invention is inserted into an expression cassette designed for the chosen host and introduced into the host where it is recombinantly produced. The choice of specific regulatory sequences such as promoter, signal sequence, 5′ and 3′ untranslated sequences, and enhancer appropriate for the chosen host is within the level of skill of the routineer in the art. The resultant molecule, containing the individual elements operably linked in proper reading frame, is inserted into a vector capable of being transformed into the host cell. Suitable expression vectors and methods for recombinant production of proteins are well known for host organisms such as E. coli, yeast, and insect cells (see, e.g., Luckow and Summers, Bio/Technol. 6: 47 (1988)). Specific examples include plasmids such as pBluescript (Stratagene, La Jolla, Calif.), pFLAG (International Biotechnologies, Inc., New Haven, Conn.), pTrcHis (Invitrogen, La Jolla, Calif.), and baculovirus expression vectors, e.g., those derived from the genome of Autographica californica nuclear polyhedrosis virus (AcMNPV). A preferred baculovirus/insect system is pVI11392/Sf21 cells (Invitrogen, La Jolla, Calif.).

Recombinantly produced polypeptide is isolated and purified using a variety of standard techniques. The actual techniques used varies depending upon the host organism used, whether the enzyme is designed for secretion, and other such factors. Such techniques are well known to the skilled artisan (see, e.g. chapter 16 of Ausubel, F. et al., “Current Protocols in Molecular Biology”, pub. by John Wiley & Sons, Inc. (1994).

Recombinantly produced polypeptides are useful for a variety of purposes. For example, they are used in assays to screen for chemicals that interact with the polypeptide or that alter the activity of the polypeptide.

V. Method to Assay a Compound that Interact with a Polypeptide of the Present Invention

In another aspect of the present invention, assays to identify a compound that interacts with a polypeptide comprising a 3′-5′ exonuclease domain are disclosed. In a preferred embodiment, such a compound is capable of altering the activity of the polypeptide. Preferably, the compound is capable of inhibiting or stimulating the activity of the polypeptide. Preferably, such compound is applied to a plant or a plant cell, and, as a result, the activity of the polypeptide in the plant or plant cell is altered. In such plant or plant cell, the expression of a nucleotide sequence of interest and as described above is altered. The present invention thus further discloses methods to alter the expression of a nucleotide sequence of interest in a plant or plant cell comprising applying to said plant or plant cell a compound capable of inhibiting the activity of a nucleotide sequence of said plant or plant cell that encodes a polypeptide comprising a 3′-5′ exonuclease domain. In a preferred embodiment, the nucleotide sequence of interest is a heterologous or an endogenous nucleotide sequence. Preferably, the plant cell comprises the heterologous nucleotide sequence as described above in section II. Preferably, the plant cell comprises a nucleotide sequence identical or substantially similar to the endogenous nucleotide sequence as described above in section II.

1. In Vitro Inhibitor Assays: Discovery of Compounds that Interacts with a Polypeptide of the Present Invention

Three methods (fluorescence correlation spectroscopy, surface-enhanced laser desorption/ionization, and biacore technologies) that can detect interactions between a polypeptide and a compound are described below.

Fluorescence Correlation Spectroscopy (FCS) theory was developed in 1972 but it is only in recent years that the technology to perform FCS became available (Madge et al. (1972) Phys. Rev. Lett., 29: 705-708; Maiti et al. (1997) Proc. Natl. Acad. Sci. USA, 94: 11753-11757). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N or C-terminus. The expression takes place in E. coli, yeast or insect cells. The protein is purified by chromatography. For example, the poly-histidine tag can be used to bind the expressed protein to a metal chelate column such as Ni2+ chelated on iminodiacetic acid agarose. The protein is then labeled with a fluorescent tag such as carboxytetramethylrhodamine or BODIPY® (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was invented by Hutchens and Yip during the late 1980's (Hutchens and Yip (1993) Rapid Commun. Mass Spectrom. 7: 576-580). When coupled to a time-of-flight mass spectrometer (TOF), SELDI provides a mean to rapidly analyze molecules retained on a chip. It can be applied to ligand-protein interaction analysis by covalently binding the target protein on the chip and analyze by MS the small molecules that bind to this protein (Worrall et al. (1998) Anal. Biochem. 70: 750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system capable to pipet the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 microlitre cell with the immobilized protein. Binding is detected by surface plasmon resonance (SPR) by recording laser light refracting from the surface. In general, the refractive index change for a given change of mass concentration at the surface layer, is practically the same for all proteins and peptides, allowing a single method to be applicable for any protein (Liedberg et al. (1983) Sensors Actuators 4: 299-304; Malmquist (1993) Nature, 361: 186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

2. In Vivo Inhibitor Assay

In another embodiment, an in vivo screening assay for compounds altering the activity of a polypeptide encoded by a nucleotide sequence of the present invention uses transgenic plants, plant tissue, plant seeds or plant cells capable of overexpressing a nucleotide sequence of the present invention.

A chemical is then applied to the transgenic plants, plant tissue, plant seeds or plant cells and to the isogenic non-transgenic plants, plant tissue, plant seeds or plant cells, and gene silencing in the transgenic and non-transformed plants, plant tissue, plant seeds or plant cells is determined after application of the chemical and compared.

VI. Assays for Testing the Alteration of Gene Silencing

Several methods are described to test for the alteration of gene silencing in a plant cell.

A. Introduction of a Marker Gene in a Plant Cell and Analysis of its Expression

A marker gene is introduced into wild-type lines and into lines with potentially altered gene silencing. An alteration in gene silencing is detected as a difference in the T1 progeny in the number of lines exhibiting low levels of marker activity vs. high levels of marker activity. Lines with high levels of marker activity are not likely to be silenced, whereas lines with low levels of or without activity are likely to be silenced. Choices for a non-endogenous marker gene include luciferase, green fluorescent protein (GFP), or beta-glucuronidase (GUS). Assay methods for each of these markers have been described (Ishitani et al. (1997) Plant Cell, 9:1935-1949; Cutler et al. (2000) Proc. Natl. Acad. Sci. USA 97: 3718-3723; Jefferson et al. (1989) EMBO J., 6:3901-3907).

B. Analysis of the Expression of an Endogenous Gene

This assay method is similar to the one above, except that an endogenous gene is used in place of a marker gene. The expression of the endogenous gene is measured in wild-type lines and in lines with potentially altered gene silencing. Both types of lines further comprise a transgenic “silencing” construct used to silence the endogenous gene. Such “silencing” construct for example comprises a promoter directing the transcription of the endogenous gene in a sense orientation, or an antisense orientation, or in both an antisense and a sense orientation in the same transcript. The promoter is for example constitutive, like ACTIN2 (An et al., 1996, Plant J., 10:107-121), or inducible, like PR1 (see e.g. U.S. Pat. No. 5,614,395), or activatable by a hybrid transcription factor (Guyer et al., 1998, Genetics 149:633-639). In plants with the “silencing” transgene, the level of gene silencing is assessed by analyzing alterations in the function of the endogenous gene, for example appearance of a mutant phenotype, relative to plants without the transgene. By comparing the range of phenotypes observed in the T1 progeny of these plants, it is determined whether the original lines have altered gene silencing capabilities. Endogenous genes that are used include for example: APETALA1, which has a mutant phenotype in which petals are absent and sepals are converted to leaves with axillary flowers (Bowman et al., 1989, Plant Cell, 1:37-52), GLABROUS1, which has a mutant phenotype in which the number of trichomes on leaves is greatly reduced (Oppenheimer et al, 1991, Cell 67:483-493), and NIM1 (also known as NPR1), which has a mutant phenotype in some ecotypes in which Peronospora isolates become infectious and SAR genes such as PR1 are not induced (for example Ryals et al. (1997) Plant Cell 9:425-39). Induction of PR1 can be detected by Northern or RT-PCR.

C. Analysis of the Expression of a Characterized Silenced Transgene

To determine whether a given line alters gene silencing, introduction of a characterized silenced (either post transcriptionally or transcriptionally) gene is accomplished by crossing the line in question with a line with a characterized silenced gene and examining the effects in the F1 and F2 progeny. For a line with a characterized silenced gene, the experiment measures changes in the levels of expression of this gene in the mutant backgrounds. For recessive mutations that might alter gene silencing, it is necessary to compare F2 progeny homozygous, heterozygous, and wild type for the mutant allele for differences in expression levels of the silenced gene. For dominant mutations that might alter gene silencing, it is possible to compare F1 progeny heterozygous and wild type for the mutant allele for differences in expression levels of the silenced gene. A line with a constitutive promoter and a marker gene is used in such experiments.

VII. Assay for 3′-5′ Exonuclease Activity

Assays are available to test for 3′-5 exonuclease activity in the polypeptides encoded by the nucleotide molecules and sequences of the present invention. Assays for 3′-5′ exonuclease activity are set forth in Kamath-Loeb et al. (1998) J. Biol. Chem. 273:34145-50, Huang et al., (1998) Nat. Genet. 20:114-6, and Suzuki et al. (1999) Nucleic Acids Res. 27:2361-8, each incorporated by reference in their entireties. Briefly, the polypeptide or protein is incubated with radioactively labeled DNA oligomers. After incubation, the reaction products are analyzed by polyacrylamide gel electrophoresis.

VIII. Polypeptides Encoded by the Nucleic Acid Molecules.

The present invention provides polypeptides encoded by the nucleic acid molecules of the invention and variants thereof. These polypeptides are exemplified by those encoded by the nucleotide sequences of SEQ ID NOS: 2, 4, 6, 22, 18, 12, 14, 24, 36 and 38; polypeptides encoded by nucleic acid sequences having at least 70% sequence similarity to the sequences of SEQ ID NOS: 1, 3, 5, 21, 9, 11, 13, 23, 35 or 37 and variants and mutants thereof. Preferably, the isolated and substantially purified polypeptides are identical or substantially similar to the amino acid sequence of SEQ ID NO:24.

The polypeptides of the invention may be altered in various ways including amino acid substitutions, deletions, truncations, and insertions. Methods for such manipulations are generally known in the art. Methods for mutagenesis and nucleotide sequence alterations are well known in the art. See, for example, Kunkel, Proc. Natl. Acad. Sci. USA, 82:488, (1985); Kunkel et al., Methods in Enzymol, 154:367 (1987); U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions may be preferred.

The proteins of the invention encompass both naturally occurring polypeptides as well as variants and modified forms thereof. Obviously, the mutations that will be made in the DNA encoding the mutation must not place the sequence out of reading frame and preferably will not create complementary regions that could produce secondary mRNA structure. See, EP Patent Application Publication No. 75,444.

The invention will be further described by reference to the following detailed examples. These examples are provided for purposes of illustration only, and are not intended to be limiting unless otherwise specified.

### EXAMPLES

### Example 1

### Identification of Polypeptides Comprising a 3′-5′ Exonuclease Domain

Method 1

Using the MEME and MotifSearch programs of GCG SEQWEB (version 1.1, University of Wisconsin), seven Arabidopsis polypeptide sequences potentially containing RNase D-related motifs are identified. MEME starts with a set of unaligned polypeptide sequences and identifies common motifs. Then, these motifs are used to create gapless profiles that can be used as input to MotifSearch to search other sequences for these motifs.

First, the C. elegans mut-7 gene (ZK1098.8, GenPept accession CAA80137) is used in a BLASTP search to identify related Arabidopsis polypeptide sequences. One polypeptide sequence is identified (GenPept accession CAB36851, SEQ ID NO:2). Second, sequences of several of the proteins in Branch B of FIG. 4 of Moser et al. (1997) (Nucl. Acids Res. 25:5110-5118) are used together with the Arabidopsis predicted polypeptide sequence (GenPept accession CAB36851, SEQ ID NO:2) to identify common motifs with the MEME program. These protein sequences include: entire C. elegans mut-7 (GenPept accession CAA80137, SEQ ID NO:16, corresponding nucleotide sequence SEQ ID NO:15), C-terminus (amino acid positions 428 to end) of C. elegans mut-7-related protein (ZK1098.3, GenPept accession CAA80141), C-terminus (amino acid positions 291 to end) of H. sapiens 100 kDa nucleolar Polymyositis Scleroderma autoantigen (PMSC100, GenPept accession CAA46904), C-terminus (amino acid positions 216 to end) of S. cerevisiae RRP6 (GenPept accession NP_{—}014643), N-terminus (amino acid positions 1 to 333) of H. sapiens Werner syndrome protein (WRN, GenPept accession AAF06162, SEQ ID NO:18, corresponding nucleotide sequence SEQ ID NO:17), entire E. coli RNase D (SwissProt accession P09155), and C-terminus (amino acid positions 546 to end) of D. melanogaster Egalitarian (EGL, GenPept accession AAB49975, and entire phage phi-C31 hypothetical protein 11 (GenPept accession CAA53907). Truncated versions of some proteins are used to allow identification of RNase D related motifs in polypeptides with other sequence regions or motifs. Third, five MEME motifs are identified. Fourth, MotifSearch is used to search GenPept Plant division for sequences containing these motifs and seven Arabidopsis polypeptide sequences are identified. The GenPept accessions for these sequences are listed from lowest to highest P-value from the MotifSearch program: CAB36851 (SEQ ID NO:2, corresponding nucleotide sequence SEQ ID NO:1), AAC69936 (SEQ ID NO:6, corresponding nucleotide sequence SEQ ID NO:5), AAD25623 (SEQ ID NO:4, corresponding nucleotide sequence SEQ ID NO:3), AAD26968 (SEQ ID NO:10, corresponding nucleotide sequence SEQ ID NO:9), AAC25931 (SEQ ID NO:12, corresponding nucleotide sequence SEQ ID NO:11), AAC42241 (SEQ ID NO:8, corresponding nucleotide sequence SEQ ID NO:7), AAF98185 (SEQ ID NO:14, corresponding nucleotide sequence SEQ ID NO:13). A lower value has greater probability of being significantly different from random.

The inventors of the present invention also discovered that the 5′ end of GenPept accession AAC42241 is missing due to incorrect annotation, and that GenPept accession AAC42241 lacks the exo I motif of the 3′-5′ exonuclease domain. The amino acid sequence comprising the entire 3′-5′ exonuclease domain (including exo 1) is disclosed for the first time in the instant application and is set forth in SEQ ID NO:22. The corresponding nucleotide sequence is set forth in SEQ ID NO:21.

Method 2

The C. elegans mut-7 protein contains a 3′-5′ exonuclease domain. The HMMsearch (hidden Markov model) program (Eddy, S. R. (1996) Curr. Opin. Struct. Biol. 6:361-365) is used to search the GenPept plant division for protein sequences with the 3′-5′ exonuclease profile, which is found in the Pfam database (A. Bateman, et al. (2000) Nucleic Acids Research, 28:263-266, incorporated herein by reference in its entirety). Pfam is a database of multiple alignments of protein domains or conserved protein regions. These alignments represent some evolutionary conserved structure that has implications for the protein's function. Profile HMMs built from the Pfam alignments are used for automatically recognizing that new proteins belong to an existing protein family, even if the sequence similarity is weak. Five Arabidopsis polypeptide sequences are identified. The GenPept accessions for these sequences are listed from lowest to highest E-value from the HMMsearch program: AAD25623, AAC69936, CAB36851, AAC42241 and AAD26968. A lower value has greater probability of being significantly different from random.

The 3′-5′ exonuclease domain consists of three sequence motifs termed Exo I, Exo II, and Exo III (Moser et al. (1997) Nucl. Acids Res. 25:5110-5118). These motifs are clustered around the active site and contain four negatively charged amino acids that serve as ligands for the two metal ions necessary for catalysis in addition to a catalytically active tyrosine.

The presence of these amino acids in SEQ ID NO:2, SEQ ID NO:4, SEQ ID NO:6, SEQ ID NO:22, SEQ ID NO:10, SEQ ID NO:12, SEQ ID NO:14, SEQ ID NO:16, and SEQ ID NO:18, and their position in the corresponding amino acid sequences is indicated in Table 1 below. The positions of the exo I, exo II, and exo III motifs in these amino acid sequences is shown in Table 2.

Method 3

Additional Arabidopsis genes that encode proteins with 3′-5′ exonuclease domains are identified starting from the experimentally determined cDNA sequence for the gene encoded by SEQ ID NO:1. This protein sequence (SEQ ID NO:24) is identified as described in Example 6 (see below). A BLASTp search (Altschul, S. F. et al. (1997) Nucleic Acids Res. 25:3389-3402) with SEQ ID NO:24 as the query reveals that there are several predicted Arabidopsis proteins with E values less than 1 E-03. From this group of Arabidopsis proteins, a HMM search is performed with each protein as a query compared to the HMM database of PFAM models (version 6.4) on a TimeLogic DeCypher machine. Two Arabidopsis proteins (AAG50917 (SEQ ID NO:36) and BAB11227 (SEQ ID NO:38)) are identified that were not identified by methods 1 and 2 earlier. The nucleotide sequences corresponding to AAG50917 and BAB11227 are SEQ ID NO:35 and SEQ ID NO:37, respectively. These proteins have E values from the HMM search that are below 1E-01 and likely to be significant. For comparison, the E-values for all the Arabidopsis proteins identified in Example 1 are shown in the third column in Tables 1 and 2 as recalculated with the same HMM program, PFAM model, and computer server.

TABLE 1

| | E-value | E-value | | | |
|---|---|---|---|---|---|
| Accession # | HMM | HMM** | exo I | exo II | exo III |
| AAD25623 | 4.6E−54 | 2.7E−67 | D140, E142 | D199 | Y264, D268 |
| (SEQ ID | | | | | |
| NO: 4) | | | | | |
| AAC69936 | 1.5E−44 | 5.8E−82 | D80, E82 | D138 | Y203, D207 |
| (SEQ ID | | | | | |
| NO: 6) | | | | | |
| CAB36851 | 2.0E−04 | 9.8E−12 | D133, E135 | D194 | Y263, D267 |
| (SEQ ID | | | | | |
| NO: 2) | | | | | |
| AAC42241* | 1.5E−01 | 7.6E−27 | D50, E52 | D108 | A192, D196 |
| (SEQ ID | | | | | |
| NO: 22) | | | | | |
| AAD26968 | 5.1E+00 | — | D61, Q63 | D118 | Q186, D190 |
| SEQ ID | | | | | |
| NO: 10) | | | | | |
| AAC25931 | — | — | G70, Q72 | D127 | Q195, D199 |
| (SEQ ID | | | | | |
| NO: 12) | | | | | |
| AAF98185 | — | — | — | —? | Y60, R64 |
| (SEQ ID | | | | | |
| NO: 14) | | | | | |
| AAG50917 | — | 6.6E−11 | D384, E386 | D449 | Y531, D535 |
| (SEQ ID | | | | | |
| NO: 36) | | | | | |
| BAB11227 | — | 2.4E−14 | D34, E36 | D104 | Y186, D190 |
| (SEQ ID | | | | | |
| NO: 38) | | | | | |
| CAA80137 | — | — | D435, E437 | D503 | Y585, D589 |
| (SEQ ID | | | | | |
| NO: 16) | | | | | |
| AAF06162 | — | — | D82, E84 | D143 | Y212, D216 |
| (SEQ ID | | | | | |
| NO: 18) | | | | | |

*using corrected sequence because 5′ end is missing due to incorrect annotation, so that exo 1 may be present.
**HMM PFAM analysis done as per Method 3 of Example 1
exo I, II, & III motifs defined as in FIG. 6 of Mian (1997) Nucleic Acids Res 25: 3187

TABLE 2

| | E-value | E-value | | | |
|---|---|---|---|---|---|
| Accession # | HMM | HMM** | exo I | exo II | exo III |
| AAD25623 | 4.6E−54 | 2.7E−67 | 136-145 | 191-206 | 261-271 |
| (SEQ ID | | | | | |
| NO: 4) | | | | | |
| AAC69936 | 1.5E−44 | 5.8E−82 | 76-85 | 130-135 | 200-210 |
| (SEQ ID | | | | | |
| NO: 6) | | | | | |
| CAB36851 | 2.0E−04 | 9.8E−12 | 129-138 | 186-201 | 260-270 |
| (SEQ ID | | | | | |
| NO: 2) | | | | | |
| AAC42241* | 1.5E−01 | 7.6E−27 | 46-55 | 100-115 | 189-199 |
| (SEQ ID | | | | | |
| NO: 22) | | | | | |
| AAD26968 | 5.1E+00 | — | 57-66 | 110-125 | 183-193 |
| SEQ ID | | | | | |
| NO: 10) | | | | | |
| AAC25931 | — | — | 66-75 | 119-134 | 192-202 |
| (SEQ ID | | | | | |
| NO: 12) | | | | | |
| AAF98185 | — | — | — | — | 57-67 |
| (SEQ ID | | | | | |
| NO: 14) | | | | | |
| AAG50917 | | 6.6E−11 | 380-389 | 441-456 | 528-538 |
| (SEQ ID | | | | | |
| NO: 36) | | | | | |
| BAB11227 | | 2.4E−14 | 30-39 | 96-111 | 183-193 |
| (SEQ ID | | | | | |
| NO: 38) | | | | | |
| CAA80137 | — | — | 431-440 | 495-510 | 582-592 |
| (SEQ ID | | | | | |
| NO: 16) | | | | | |
| AAF06162 | — | — | 78-87 | 135-150 | 209-219 |
| (SEQ ID | | | | | |
| NO: 18) | | | | | |

*using corrected sequence because 5′ end is missing due to incorrect annotation, so that exo 1 may be present.
**HMM PFAM analysis done as per Method 3 of Example 1

### Example 2

### Insertion Mutagenesis in a Nucleotide Sequence Encoding a Polypeptide Comprising a RNase D Related Domain

Insertion mutagenesis facilitates direct reverse genetic screens by providing a physical link to the gene of interest. In plants both T-DNA and transposon insertion mutagens have been utilized as insertion mutagens (Winkler et al. (1998) Methods Mol. Biol. 82:129-136, Martienssen (1998) PNAS 95:2021-2026). T-DNA insertions within any given gene can be detected by polymerase chain reaction (PCR) methods utilizing one gene specific primer and one T-DNA specific primer (Winkler et al. (1998) Plant Physiol. 3:743-750, and Krysan et al. (1999) Plant Cell, 11:2283-2290). Specific PCR product is formed only when a T-DNA element has inserted either within or close to the gene of interest. Due to the exponential nature of PCR amplification, it is possible to screen many thousands of independently transformed Arabidopsis mutants by sample pooling (Krysan et al., 1999). Once a T-DNA pool is identified with an insertion in the gene of interest, the process of isolating a single plant with that insertion requires de-convolution of the pool architecture.

To assess the function of a polypeptide encoded by the nucleotide sequence set forth in SEQ ID NO:1, a pool of ˜60,480 independent tagged Arabidopsis lines (Krysan et al., 1999) is screened by PCR utilizing pairs of primers corresponding to the T-DNA left border and the SEQ ID NO:1 3′-specific region. The SEQ ID NO:1 3′ specific primer (5′-cga cat gat ctg ata cat cgt tat gcc att-3′, SEQ ID NO:19) corresponds to position 96817-96790 on BAC F18A5, GenBank accession number AL035528.2. The left border primer from A. tumefaciens T-DNA vector pD991 is represented by SEQ ID NO:20 (5′-cat ttt ata ata acg ctg cgg aca tct ac-3′). (Krysan et al., 1999). One specific PCR product is identified, isolated and designated S11.13. Sequencing of the PCR-amplified fragment reveals a T-DNA insertion 26 bp 5′ of the predicted CDS region of SEQ ID NO:1. De-convolution of pool architecture as described (Krysan et al., 1999) leads to the identification of seven individual lines containing the specific T-DNA element, designated S11.13-8, S11.13-13, S11.13-34, S11.13-38, S11.13-41, S11.13-44, S11.13-48. PCR is subsequently utilized for genotyping individual lines. All of the lines are heterozygous for the insertion, except S11.13-34 is homozygous for the insertion. No visible phenotype is observed in line S11.13-34 at the embryo and seedling stages.

### Example 3

### Analysis of the Expression of a Characterized Silenced Transgene in Arabidopsis Line S11.13-34

Line S11.13-34 (see Example 1 above) is crossed with line L1, which has been shown to have a post-transcriptionally silenced GUS transgene (Elmayan et al. (1998) Plant Cell 10:1747-1758). Individual F1 progeny with a silenced GUS transgene are allowed to self fertilize. About 100 F2 progeny from individual F1 plants are grown and tested for GUS activity. The genotype of each F2 plant with respect to the T-DNA insertion in the RNase D related domain (RDRD) gene is determined by PCR as described in Example 2. Similarly, the presence of the GUS transgene is determined by PCR for each plant. Levels of GUS activity in plants homozygous for the insertion in the RNase D related domain gene are compared to plants heterozygous for the insertion RDRD gene and wild-type plants.

### Example 4

### The Arabidopsis thaliana Transgenic Lines 8Z-2 and 5 Exhibit Post-Transcriptional Silencing of a Green-Fluorescent Protein Reporter Gene

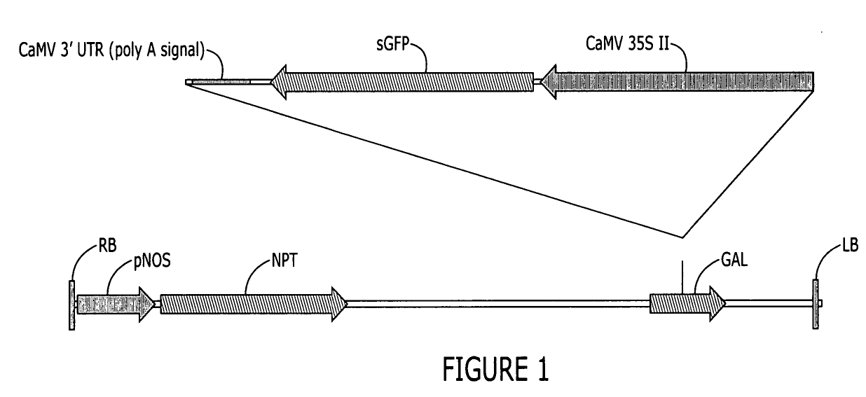

Agrobacterium-mediated transformation as described by Bechtold (Methods in Molecular Biology, 82: 259-266, 1998) is used to obtain transgenic Arabidopsis thaliana ecotype Columbia plants exhibiting PTGS. The Ti-plasmid used contains a chimeric green fluorescent protein (GFP) (Reichel et al. (1996) PNAS 93: 5888-93) reporter gene regulated by a duplicated cauliflower mosaic virus (CaMV) 35S RNA promoter and transcriptional terminator (Goodall and Filipowicz (1989) Cell 58: 473-483) in the binary vector pBIN19 (Bevan (1984) Nucl. Acids Res. 12: 8711-8721). The T-DNA region of this plasmid (p35S-GFP) is shown schematically in FIG. 1.

To evaluate PTGS in the resultant ³⁵S-GFP transformants, GFP expression is monitored in transgenic plants by GFP excitation with UV light (approximate range of wavelengths 390 to 480 nm). Selection of transgenic lines showing PTGS is based on absence of GFP expression in mature plants that showed normal GFP expression in earlier stages of plant development. Based on this criterion, two lines designated as 8Z-2 and 5, which are homozygous for the T-DNA insert, show PTGS associated with greatly reduced GFP-mRNA levels detected by RNA blot hybridization as described by Sambrook et al. (Molecular Cloning, 2^{nd} edition. 1989). Line 8Z-2 shows PTGS in approximately 90-96% of sibling plants. Line 5 shows PTGS in approximately 30-50% of sibling plants.

DNA blot hybridization as described by Sambrook et al. (Molecular Cloning. 2^{nd} edition, 1989) reveals that post-transcriptionally silenced line 8Z-2 carries two copies of T-DNA. Further analysis based on polymerase chain reaction (PCR) and utilizing combinations of T-DNA specific primers (Kumar and Fladung (2000) BioTechniques 28: 1128-1137) shows that these two copies are arranged in a direct tandem repeat. Similarly, line 5 is shown to carry one full-length T-DNA and a second, truncated T-DNA copy arranged in an inverted tandem repeat. The genomic position of the T-DNA copies in line 8Z-2 is determined to be chromosome I, BAC F22L4, gene #11 by thermal asymmetric interlaced polymerase chain reaction (TAIL-PCR) (Liu et al. (1995) Plant Journal 8: 457-463) using the T-DNA specific primers LB1 (5′-ttc gga acc acc atc aaa cag g-3′, SEQ ID NO:25), LB2 (5′-ttg ctg caa ctc tct cag ggc c-3′, SEQ ID NO:26), and LB3 (5′-tca gct gtt gcc cgt ctc act-3′, SEQ ID NO:27) and the degenerate primer AD3 (5′-wgt gna gwa nca nag a-3′, where W=A/T and N=G/A/T/C, SEQ ID NO:28). The genomic position of the T-DNA copies in line 5 is determined to be linked to BAC F22L4 on chromosome 1.

### Example 5

### Analysis of the Expression of the Silenced 8Z-2 Transgene in Arabidopsis Line S11.13-34

The line 8Z-2 (see Example 4 above) is crossed with the line S11.13-34 (see Example 2 above) and the resultant F1 generation plants are allowed to self-fertilize to obtain the F2 generation. Approximately 60 F2 plants are grown and tested for a presence of T-DNA insertion in the RDRD gene derived from the S11.13-34 parental line and for the 35S-GFP T-DNA derived from the 8Z-2 parental line. The presence of the T-DNA insertion in the RDRD gene is demonstrated as described in Example 2. Plants homozygous for this T-DNA insertion are then checked for homozygosity by PCR using the 3′ specific primer (SEQ ID NO:19) and 36851TD#3 (5′-gct ccg ccc aca taa ttc aaa caa cac-3′, SEQ ID NO:29). These primers span a region of genomic DNA including the insertion site such that only the wild-type copy of DNA results in amplification of a genomic fragment. A similar strategy is used to screen for lines homozygous for the 35S-GFP T-DNA. First, the presence of the ³⁵S-GFP T-DNA is demonstrated by using the T-DNA-specific PCR primer LB1 and the gene-specific PCR primer L22F4F (5′-ttc gaa aac att acc tcc gat c-3′, SEQ ID NO:30). Second, plants carrying the 35S-GFP T-DNA are tested for homozygosity by using the gene-specific primers L22F4F and F22L4R (5′-ggc ttt tgc att tgg tat cta cta g-3′, SEQ ID NO:31) The plants homozygous for both the S11.13-34 and 8Z-2 transgenes and plants homozygous for the 8Z-2 transgene but with no S11.13-34 transgene are allowed to self fertilize to obtain F3 generation plants. These plants and the parental line 8Z-2 are scored for incidence of PTGS based on GFP fluorescence as described in Example 4. The results summarized in Table 3 show that PTGS of the 35S-GFP transgene is lost in plants with a T-DNA insert interrupting the region encoding a polypeptide comprising an RNase D-related domain.

TABLE 3
The Incidence of 35S-GFP PTGS in S11.13-34 × 8Z-2 Hybrids

| | | | % Plants | Total number |
|---|---|---|---|---|
| | | | exhibiting | of plants |
| Line tested | Description | Comments | PTGS | scored |
| Parental 8Z-2 | Homozygous for the 35S-GFP | Gene encoding the RNase D | 90 | 40 |
| | transgene. No S11.13-34 | domain related protein is | | |
| | T-DNA insert | expressed. | | |
| Outcrossed F3 | F3 plants derived from | Gene encoding the RNase D | 88 | 33 |
| | the S11.13-34 × 8Z-2 F3 hybrid | domain related protein is | | |
| | homozygous for the 35S-GFP | expressed. | | |
| | transgene and the S11.13-34 | | | |
| | T-DNA crossed out | | | |
| Homozygous F3 | F3 plants derived from | Gene encoding the RNase D | 0 | 36 |
| | the S11.13-34 × 8Z-2 F3 hybrid | domain related protein with | | |
| | homozygous for the 35S-GFP | T-DNA insertion. mRNA is not | | |
| | and S11.13-34 T-DNAs | expressed. | | |

### Example 6

### Expression of RNase D Domain Related Protein mRNAs in Arabidopsis Line S11.13-34

The accumulation of RDRD gene mRNA is measured by RT-PCR. The primers AtWRN CDS F (5′-atg tca tcg tca aat tgg atc gac g-3′, SEQ ID NO:32) and AtWRN-RT_R (5′-cgc tta tca acc tca gta gca gtc ttg-3′, SEQ ID NO:33) are designed to amplify a 329 bp fragment spanning a 5′ fragment of the coding sequence. The fragment of predicted length is detected in RNA samples prepared from wild-type Arabidopsis plants. Neither this predicted fragment nor any other sequences are detected in RNA samples prepared from the S11.13-34 mutant. This indicates that RDRD mRNA is expressed in wild-type plants, but not in the homozygous RDRD mutant S11.13-34.

### Example 7

### Identification of a cDNA Sequence Encoding a Polypeptide Comprising a RNase D Related Domain

The gene encoding SEQ ID NO:1, which is also known as AT4g13870 located on Arabidopsis thaliana chromosome IV contig fragment 37 (ATCHRIV37, GenBank accession AL161537), encodes a polypeptide comprising a RNase D related domain. The cDNA for this gene is isolated as follows. 5′ and 3′ RACE primers are designed based on the exon/intron boundaries in the gene model in ATCHRIV37. 5′ and 3′ RACE is performed (GeneRacer kit, Clontech). The resulting amplicons are TA-cloned (Original TA-Cloning kit, Invitrogen) and sequenced. The elucidated cDNA sequence (SEQ ID NO:23) differs from the sequence predicted in the GenBank annotation, thus identifying the actual open reading frame. SEQ ID NO:24 contains the protein sequence predicted from a translation of bases 42 to 905 of this cDNA. Analysis of the cDNA sequence from this gene reveals a high degree of similarity to an Arabidopsis thaliana mRNA for an exonuclease named “wrnexo” (GenBank accession AJ404476). The cloned cDNA sequence is nearly identical to that of wrnexo. The two sequences are likely to derive from the same gene. The difference between the two sequences is noted in 9 extra bases, present in the cloned cDNA encoding a polypeptide comprising a RNase D related domain (bases 830 to 838 of SEQ ID NO:23) but absent in wrnexo.

### Example 8

### Overexpression of a Nucleotide Sequence Encoding a Polypeptide Comprising a RNase D Related Domain

A transgenic construct designed to overexpress a polypeptide comprising a RNase D related domain is introduced into a transgenic line comprising a second transgene. A suitable line expresses the second transgene at a high level with no silencing or without complete silencing, preferably with less than half the plants showing silencing or with the silenced plants showing silencing to levels greater than 50% of the average levels of all the plants. The transgenic construct is created by expressing the GUS marker gene (GenBank accession S69414), using the strong constitutive ACT2 promoter (GenBank accession U41998), with the CaMV 35S transcriptional terminator (nucleotides 2868 to 2938 in pJG304 (Guyer et al., 1998, Genetics 149:633-639)) in a binary T-DNA vector. This construct is introduced into Arabidopsis via agrobacterium-mediated transformation. T2 plants from a single T1 plant expressing high levels of GUS activity are examined for silencing.

These T2 plants, or their progeny, are also transformed with one of two constructs. One construct allows overexpression of the RNase D related domain coding sequence (bases 42 to 908 of SEQ ID NO:23) with a strong promoter and a transcriptional terminator different from those used in the construct described above. The other construct is a control that is essentially the same as the RNase D related domain construct, except that in place of a RNase D related domain protein, a marker gene, such as luciferase or GFP is overexpressed or no gene is overexpressed. These two binary vector constructs have a selectable marker that differs from the GUS construct, so that they can be used to superinfect with a second T-DNA construct. When each of these constructs is transformed into the T2 plants described above, the level of GUS expression is determined for the doubly-transformed T1 progeny. Those T1 plants overexpressing the RNase D related domain protein are expected to have lower levels of GUS expression due to increased silencing. If a difference is not detected in those T1 plants, lines homozygous for the RNase D related domain overexpression construct can be produced in the T2 generation and examined. Alternatively, a nucleotide sequence set forth in any one of SEQ ID NO:3, SEQ ID NO:5, SEQ ID NO:21, SEQ ID NO:9, SEQ ID NO:11, SEQ ID NO:13, SEQ ID NO:15, SEQ ID NO:17, SEQ ID NO:35 or SEQ ID NO:37 is included in a construct as described above and is used for overexpression of a polypeptide comprising a 3′-5′ exonuclease domain.

### Example 9

### Complementation of the PTGS Deficiency of Line S11.13-34 by Overexpression of a Nucleotide Sequence Encoding RDRD Confirms that a Polypeptide Comprising an RNase D Related Domain is Required for PTGS

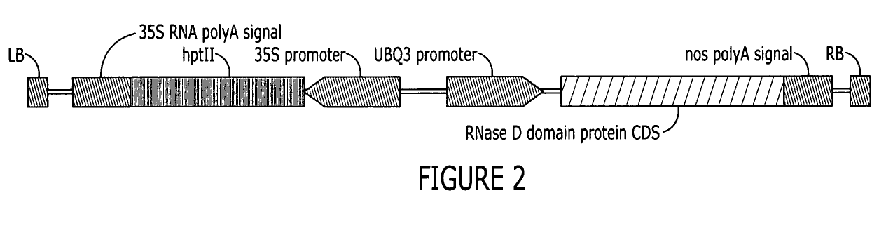

A construction designed to overexpress a polypeptide comprising an RNase D related domain is introduced into Arabidopsis plants as described in Example 4. The coding sequence comprising an RNase D related domain is amplified by RT-PCR from RNA prepared from Arabidopsis leaves using the primers AtWRN CDS F (SEQ ID NO:32) and AtWRN CDS R (5′-tta tga gcc act gac agc atc agg-3′) (SEQ ID NO:34). This RDRD coding sequence was placed under the regulation of the strong, constitutive UBQ3 gene promoter (BAC F15A17, GenBank accession AL163002) in binary vector pCAMBIA-1380 (GenBank accession AF234301). The resultant RDRD expression vector pRDP1 is shown schematically in FIG. 2.

For complementation studies, RDP1 transformants obtained by transformation of wild-type Arabidopsis plants with the vector pRDP1 are allowed to self-fertilize. The resultant T1 generation plants are tested for the hygromycin resistance phenotype to detect the presence of the RDP1 T-DNA. The hygromycin-resistant plants are then allowed to self fertilize and the resultant T2 generation is scored for hygromycin-resistance to identify homozygous transformants with T-DNA inserts at a single locus. Homozygous RDP1 plants are crossed with the double-homozygous F3 generation 8Z-2 S11.13-34 transformants described in Example 5 to obtain the F1 generation. F1 plants are allowed to self-fertilize and the resultant F2 generation plants resistant to both kanamycin and hygromycin are allowed to self-fertilize to obtain the F3 generation. The F3 plants are screened for antibiotic resistance to identify plants homozygous for the RDP1, 35S-GFP, and S11.13-34 T-DNAs. These triple-homozygous lines, the homozygous parent 8Z-2 S11.13-34 line, and the homozygous 8Z-2 35S-GFP line are screened for PTGS of the 35S-GFP transgene. Restoration of PTGS in the triple-homozygous line expressing the uninterrupted RDRD coding region in pRDP1 indicates that expression of an intact RDRD gene can complement the deficiency in PTGS in the S11.13-34 knockout line. This, together with the expression studies shown in Example 6 confirms that expression of the RDRD gene is required for PTGS.

### Example 10

### Overexpression of RDRD in Arabidopsis Promotes PTGS

Homozygous RDP1 transformants (see Example 9) are crossed with PTGS lines 8Z-2 and 5 to obtain F3 generation plants homozygous for both the 35S-GFP and RDP1 transgenes by using the methods described in Example 9. Assaying these homozygous lines for GFP expression as described in Example 4 shows that there is an increase in the fraction of plants exhibiting PTGS among the 8Z-2 RDP and 5 RDP plants compared with the original 8Z-2 and 5 lines, respectively. Therefore, overexpression of additional copies of a RDRD transgene promotes PTGS of lines showing less than a 100% incidence of PTGS.

The present invention is not to be limited in scope by the specific embodiments described which are intended as single illustrations of individual aspects of the invention, and any constructs, nucleic acid sequences or transformed plants which are functionally equivalent are within the scope of this invention. Indeed, various modifications of the invention in addition to those shown herein will become apparent to those skilled in the art from the foregoing description and accompanying drawings. Such modifications are intended to fall within the scope of the appended claims.

Various patents and references are cited within the present specification, all of which are incorporated by reference in their entireties.

What is claimed is:

1. A transgenic plant cell comprising an endogenous nucleotide sequence identical to SEQ ID NO:1 and wherein said plant cell comprises a mutation in said endogenous nucleotide sequence, or in a regulatory region thereof.

2. The transgenic plant cell of claim 1, wherein the mutation is due to an insertion of a nucleic acid molecule.

3. The transgenic plant cell according to claim 1, wherein said first DNA molecule the insertion of a nucleic acid molecule comprises one T-DNA border region.

4. The transgenic plant cell according to claim 3, wherein said first DNA molecule the insertion comprises a transposable element.

5. A transgenic plant or progeny thereof, or seeds thereof comprising the plant cell of claim 1.

6. A transgenic plant or progeny thereof, or seeds thereof comprising the plant cell of claim 2.

7. A method for altering the expression in a plant cell or plant of an endogenous nucleotide sequence encoding a polypeptide of SEQ ID NO:2, wherein reducing the transcription or translation of said endogenous nucleotide sequence in the plant cell or plant comprises the step of:
modifying by insertional mutagenesis in said plant cell or plant at least one chromosomal copy of the nucleotide sequence encoding a polypeptide of SEQ ID NO:2 or of a regulatory region thereof.

8. The method of claim 7 wherein the endogenous nucleotide sequence encoding the polypeptide of SEQ ID NO:2 is nucleotide sequence of SEQ ID NO:1.

9. A method for increasing the expression of a nucleotide sequence of interest in a plant cell or plant comprising the steps of:
a) decreasing the expression in said plant cell or plant of an endogenous nucleotide sequence of said plant cell encoding a polypeptide of SEQ ID NO:2 by modifying by insertional mutagenesis in said plant cell both chromosomal copies of the nucleotide sequence encoding the polypeptide of SEQ ID NO:2 or a regulatory region thereof; and
b) introducing into said plant cell or plant a nucleic acid molecule comprising said nucleotide sequence of interest, wherein the expression of said nucleotide sequence of interest in said plant cell or plant cell is increased.

10. The method according to claim 9, wherein said endogenous nucleotide sequence comprises SEQ ID NO:1.

11. The transgenic plant cell of claim 1, wherein the mutation is a deletion or rearrangement.

12. The transgenic plant cell of claim 1, wherein the mutation is a point mutation.

13. The transgenic plant cell of claim 1, wherein the endogenous nucleotide sequence is a DNA molecule comprising the nucleotide sequence of SEQ ID NO:1 and comprises a mutation.
